(19) United States
(12) Patent Application Publication
(10) Pub. No.: US 2010/0048920 A1
(43) Pub. Date: Feb. 25, 2010

(54) Preparation of (+)-Catechin, (-)-Epicatechin, (-)-Catechin, and (+)-Epicatechin and Their 5,7,3',4'-Tetra-O-Benzyl Analogues

(75) Inventors: Leo J. Romanczyk, JR., Hackettstown, NJ (US); Pradeep K. Sharma, Westford, MA (US); Daming Gou, Burlington, MA (US); Yanni Gou, Acton, MA (US)

Correspondence Address: GIBBONS P.C., ONE GATEWAY CENTER, NEWARK, NJ, 07102, US

(73) Assignees: Mars, Incorporated, Mclean, VA (US); Johnson Matthey Pharmaceutical Materials, Inc., Wayne, PA (US)

(21) Appl. No.: 11/993,141

(22) PCT Filed: Jun. 28, 2006
(86) PCT No.: PCT/US06/25482

§ 371 Date: Dec. 19, 2007

Related U.S. Application Data
(60) Provisional application No. 60/695,031, filed on Jun. 29, 2005.

(51) Int. Cl. C07D 311/02; C07C 45/78

(52) U.S. Cl. 549/399; 568/312

(57) ABSTRACT

Processes for preparing racemic mixtures of 5,7,3′,4′-tetra-O-benzyl-(±)-catechin and (±)-epicatechin involves (i) condensing 2-hydroxy-4,6-bis(benzyloxy)-acetophenone and 3,4-bis(benzyloxy)benzaldehyde, cyclizing the resulting compound, oxidizing the resulting compound; (ii) dihydroxylating (E)-3-(3′,4′-bis(benzyloxy)phenyl)prop-2-ene-1-ol and reducing the 1,2-diol; or (iii) coupling 3,5-bis(benzyloxy)phenol with (E)-3,5-bis(benzyloxy)-2-(3′,4′-bis(benzyloxy)phenyl)allyl)phenol and cyclizing the resulting chalcone.

A process for preparing the benzylated epimers of catechin and epicatechin involves seven steps. 3,4-Bis(benzyloxy)benzaldehyde is coupled with 2-hydroxy-4,6-benzyloxy-acetophenone to form a chalcone. The chalcone is selectively reduced to an alkene. The phenolic group of the alkene is protected. The protected alkene is asymetrically dihydroxylated. The resulting compound is deprotected, cyclized, and finally hydrolyzed.

Epimers resulting from these processes are chemically resolved or separated by chiral high pressure liquid chromatography.

Also disclosed is a method for preparing enantiomerically pure 5,7,3′,4′-tetra-O-benzyl-(+)-catechin from a racemic mixture using dibenzoyl-L-tartaric acid monomethyl ester.

Further, disclosed is an improved process for preparing dibenzoyl-L-tartaric acid monomethyl ester.

### CROSS REFERENCE TO RELATED APPLICATIONS

This application is a PCT application which claims priority to provisional application Ser. No. 60/695,031 filed Jun. 29, 2005 for “Synthesis and Purification of 5,7,3′,4′-Tetra-O-benzyl-(+)-Catechin”.

### BACKGROUND OF THE INVENTION

1. Field of the Invention

The invention relates to processes for the preparation and purification of 5,7,3′,4′-tetra-O-benzyl-(+)-catechin, -(−)-epicatechin, -(−)-catechin, and -(+)-epicatechin and for their debenzylation to (+)-catechin, (−)-epicatechin, (−)-catechin, and (+)-epicatechin.

2. Discussion of the Related Art

Recent studies have reported the biological activity of polyphenols such as catechin and epicatechin, their derivates such as epicatechin gallate and epigallocatechin gallate, and their oligomers, which are referred to as procyanidins.

Catechin, epicatechin, and procyanidins are naturally occurring polyphenolics that are widely distributed in the plant system. They are found in cocoa, tea, fruits, vegetables, and pine bark. As an example, green tea leaves contain (−)-epicatechin, (+)-catechin, epigallocatechin, epicatechin gallate, and epigallocatechin gallate which comprise up to 30 wt. % of the dry leaves. Their reported biological activities include anti-tumor activity, anti-mutagenic activity, and antioxidant activity. (+)-Catechin, (−)-epicatechin, (−)-catechin, and (+)-epicatechin are flavan-3-ols which have the structures shown below.

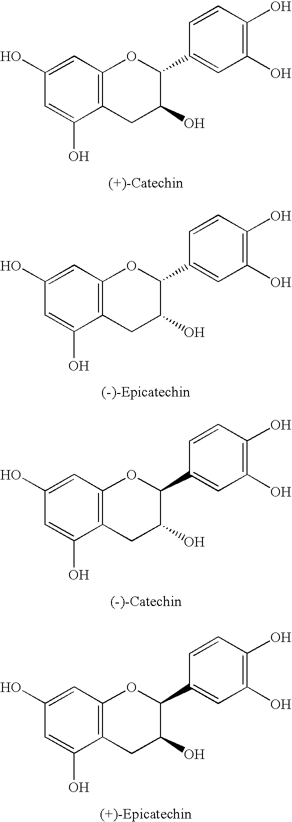

(+)-Catechin and (−)-epicatechin are the most abundant naturally occurring epimers. Oligomers of catechin and/or epicatechin are referred to as procyanidins. The monomeric units in linear procyanidins generally have (4β,8) or (4β,6)-linkages.

Processes for synthesizing (4β,8) and (4β,6) procyanidins are disclosed in U.S. Pat. No. 6,207,842 issued Mar. 27, 2001 to L. J. Romanczyk, Jr., et al., and related patents U.S. Pat. No. 6,420,572 issued Jul. 16, 2002, U.S. Pat. No. 6,528,664 issued Mar. 4, 2003, and U.S. Pat. No. 6,849,749 issued Feb. 1, 2005. Alternative processes for preparing (4β,8) and (4β,6) procyanidins are disclosed in U.S. Pat. No. 6,864,377 issued Mar. 8, 2005 to L. J. Romanczyk, Jr., et al. and related patent U.S. Pat. No. 7,015,338 issued Mar. 21, 2006.

Improved processes for preparing epicatechin-(4β,8)-catechin or -epicatechin oligomers are disclosed in U.S. 2004/0116718 published Jun. 17, 2004 and U.S. 2005/0020512 published Jan. 27, 2005 by Allan P. Kozikowski et al.

Processes for preparing novel procyanidins having (8,8), (6,6) or (6,8) linkages are disclosed in U.S. Pat. No. 6,156,912 issued Dec. 5, 2000 to Werner Tückmantel et al. An alternative synthesis for preparing procyanidins with these linkages is disclosed in U.S. Pat. No. 6,864,377 cited above.

A process for preparing novel procyanidins having (4α,8) linkages is disclosed in U.S. Pat. No. 6,476,241 issued Nov. 5, 2002 to Allan P. Kozikowski, et al. and related patent U.S. Pat. No. 6,720,432 issued Apr. 13, 2004.

To perform detailed biological studies of procyanidins and their derivatives there is a need for efficient synthetic methods for the large scale production of catechin and epicatechin monomers and their benzylated precursors from commercially available materials at the purity levels required for scale-up syntheses.

### BRIEF SUMMARY OF THE INVENTION

A process for preparing a racemic mixture of benzyl-protected epimers consisting essentially of 5,7,3′,4′-tetra-O-benzyl-(±)-catechin and 5,7,3′,4′-tetra-O-benzyl-(±)-epicatechin comprises the steps of: (a) condensing 2-hydroxy-4,6-bis(benzyloxy)-acetophenone with 3,4-bis-(benzyloxy)benzaldehyde in the presence of a base to form (E)-1-(2,4-bis(benzyloxy)-6-hydroxyphenyl-3-(3′,4′-bis(benzyloxy)phenyl)prop-2-en-1-one;

(b) cyclizing the compound formed in step (a) under reductive conditions to form 5,7-bis(benzyloxy)-2-(3′,4′-bis(benzioxy)phenyl-2H-chromene;

(c) oxidizing the compound formed in step (b) to form the racemic mixture; and (d) optionally chemically resolving the racemic mixture from step (c) or chirally separating the racemic mixture from step (c) by preparative high pressure liquid chromatography to recover the benzyl-protected epimers. The epimers are debenzylated with excess palladium hydroxide in ethyl acetate under a hydrogen atmosphere, preferably for about 2 to about 3 hours using a balloon.

An improved process for preparing (E)-1-(2,4-bis(benzyloxy)-6-hydroxyphenyl-3-(3′,4′-bis-(benzyloxy)phenyl)prop-2-en-1-one comprises the step of condensing a 2-hydroxy-4,6-bis(benzyloxy)-acetophenone with 3,4-bis-(benzyloxy)benzaldehyde in the presence of sodium hydride in N,N-dimethylformamide followed by reaction with sodium borohydride and cerium heptahydrate at a low temperature in a solution of ethanol and tetrahydrofuran. The yield is about 35-40%.

The 5,7-bis(benzyloxy)phenyl-2H-chromene formed in step (b) is a novel compound. It is prepared by cyclizing the (E)-1-(2,4-bis(benzyloxy)-6-hydroxyphenyl-3-(3′,4′-bis(benzyloxy)phenyl)prop-2-en-1-one under reductive conditions.

An alternative process for preparing a racemic mixture consisting essentially of 5,7,3′,4′-tetra-O-benzyl-protected-(O)-catechin as the major diastereomer and (±)-epicatechin as the minor diastereomer comprises the steps of:

(a) dihydroxylating 5,7-bis(benzyloxy)-2-bis(benzyloxy)phenyl-2H-chromene to form racemic 5,7-bis(benzyloxy)-2-(3′,4′-bis(benzyloxy)-phenyl)chroman-3,4-diol;

(b) reducing the racemic 3,4-diol from step (a) to form the racemic mixture; and

(c) optionally chemically resolving or chirally separating the benzylated (±)-epicatechin and the (±)-catechin in the racemic mixture. The separated epimers are debenzylated by reaction with excess palladium hydroxide in ethyl acetate under a hydrogen atmosphere, preferably using a balloon at room temperature.

Another process for preparing a racemic mixture containing 5,7,3′,4′-tetra-O-benzyl-(−)-catechin comprises the steps of:

(a) coupling 3,5-bis(benzyloxy)phenol with (E)-3-(3,4-bis(benzyloxy)-phenyl)prop-2-ene-1-ol under acidic conditions to form a mixture consisting essentially of (E)-(3,5-bis(benzyloxy)-2-(3-(3,4-bis(benzyloxy)-phenyl)allyl)-phenol;

(b) isolating the compound formed in step (a) by silica gel column chromatography;

(c) reacting the isolated compound from step (b) with tert-butyldimethylsilane chloride to form (E)-(3,5-bis(benzyloxy)-2-(3-(3′,4′-bis(benzyloxy)phenyl)allyl)-phenoxy (tert-butyl)dimethylsilane;

(d) dihydroxylating the compound from step (c) by reaction with osmium tetroxide and N-methylmorpholine-N-oxide to form racemic 3-(2,4-bis(benzyloxy)-6-(tert-butyldimethylsilyloxy)phenyl-1-(3′,4′-bis(benzyloxy)phenyl-propane-1,2-diol which upon reaction with n-tetrabutylammonium fluoride produces 3-(2,4-bis(benzyloxy)-6-hydroxyphenyl)-1-(3′,4′-bis(benzyloxy)phenyl)propane-1,2-diol;

(e) converting the 1,2-diol formed in step (d) to 3,5-bis(benzyloxy)-2-(5-(3′,4′-bis(benzyloxy)phenyl)-2-ethoxy-1,3-dioxolane-4-yl)phenol using triethylorthoformate or 3,5-bis(benzyloxy)-2-((5-(3′,4′-bis(benzyloxy)phenyl)-2-ethoxy-1,3-dioxolane-4-yl)propyl)phenol using triethylorthopropionate under acid catalyzed conditions; and

(f) treating the compound formed in step (e) with potassium carbonate in a mixture of methanol and dichloromethane or dichloroethane first at room temperature and then at about 40° to about 60° C. to form 5,7,3,4-tetra-O-benzyl-(±)-catechin and (±)-epicatechin. The solvent is removed under vacuum. The residue is extracted with ethyl acetate and water. The water is removed and the ethyl acetate is dried over sodium sulfate. The solvent is evaporated to yield crude 5,7,3′,4′-tetra-O-benzyl-(+)-catechin. The diastereomers are separated and debenzylated by reaction with palladium hydroxide in ethyl acetate at room temperature under a hydrogen atmosphere, preferably with a balloon.

A process for preparing the uncommon epimers (−)-catechin and (+)-epicatechin and their benzylated analogues comprises the steps of:

(a) condensing 2-hydroxy-4,6-bis(benzyloxy)-acetophenone with 3,4-bis(benzyloxy)benzaldehyde in the presence of a base, preferably sodium hydride, in N,N-dimethylformamide to form (E)-1-(2,4-bis(benzyloxy)-6-hydroxyphenyl-3-(3′,4′-bis(benzyloxy)phenyl)prop-2-en-1-one;

(b) selectively reducing the compound formed in step (a) with sodium borohydride and cerium chloride heptahydrate in a mixture of tetrahydrofuran and ethanol to form (E)-3,5-bis(benzyloxy)-2-(3-(3′,4′-bis(benzyloxy)phenyl)allyl)-phenol;

(c) reacting the compound formed in step (b) with tert-butyldimethylsilyl chloride in imidazole and N,N-dimethylformamide or tert-butyldimethylsilyl chloride in triethylamine and N,N-dimethylaminopyridine in dichloromethane at room temperature to form (E)-(3,5-bis(benzyloxy)-2-(3-(3′,4′-bis(benzyloxy)-phenyl)allyl)phenoxy)(tert-butyl)dimethylsilane;

(d) asymetrically dihydroxylating the compound formed in step (c), in the presence of methanesulfonamide in a mixture of tert-butanol, water, and tetrahydrofuran or dichloromethane, with AD-mix-α to form (1S,2S)-3-(2,4-bis(benzyloxy)-6-tert-(butyldimethylsiloxy)phenyl-1-(3′,4′-bis(benzyloxy)-phenyl)propane-1,2-diol or with AD-mix-β to form (1R,2R)-3-(2,4-bis(benzyloxy)-6-tert-(butyldimethylsiloxy)-phenyl-1-(3′,4′-bis(benzyloxy)phenyl)propane-1,2-diol;

(e) deprotecting the (1S,2S)- or (1R,2R)-1,2-diol formed in step (d) by reaction with n-tetrabutylammonium fluoride in acetic acid and tetrahydrofuran or dichloromethane to form (1S,2S)-3-(2,4-bis(benzyloxy)-6-(hydroxyphenyl)-1-(3′,4′-bis(benzyloxy)phenyl)propane-1,2-diol when the (1S,2S)-1,2-diol is reacted or (1R,2R)-3-(2,4-bis(benzyloxy)-6-(hydroxyphenyl)-1-(3′,4′-bis(benzyloxy)phenyl)-propane-1,2-diol when the (1R,2R)-1,2-diol is reacted; (f) reacting the deprotected (1S,2S)- or (1R,2R)-1,2-diol formed in step (e) with triethylorthopropionate or triethylorthoformate and pyridinium p-toluenesulfonate to form 5,7,3′,4′-tetra-O-benzyl-(+)-catechin-3-O-propyl ester or 5,7,3′,4′-tetra-O-benzyl-(+)-catechin-3-O-formyl ester when the (1S,2S)-1,2-diol is reacted or 5,7,3′,4′-tetra-O-benzyl-(+)-catechin-3-O-propyl ester or 5,7,3′,4′-tetra-O-benzyl-(−)-catechin-3-O-formyl ester when the (1R,2R)-1,2-diol is reacted;

(g) reacting the 5,7,3′,4′-tetra-O-benzyl-(+)-catechin-3-O-propyl ester or 5,7,3′,4′-tetra-O-benzyl-(−)-catechin-3-O-formyl ester formed in step (f) with potassium carbonate in a mixture of methanol and dichloromethane or dichloroethane to form the 5,7,3′,4′-tetra-O-(+)-catechin or 5,7,3′,4′-tetra-O-(−)-catechin; and

(h) optionally debenzylating the compound from step (g) with excess palladium hydroxide in ethyl acetate at room temperature under hydrogen atmosphere using a balloon to form (−)-catechin or (+)-catechin.

### DESCRIPTION OF THE PREFERRED EMBODIMENTS

### Part A

### Preparation of a Racemic Mixture of 5,7,3′,4′-Tetra-O-benzyl-(O)-catechin and —(±)-epicatechin from 2-Hydroxy-4,6-bis(benzyloxy)-acetophenone and 3,4-Bis(benzyloxy)benzaldehyde

The reaction sequence for this process is set out below.

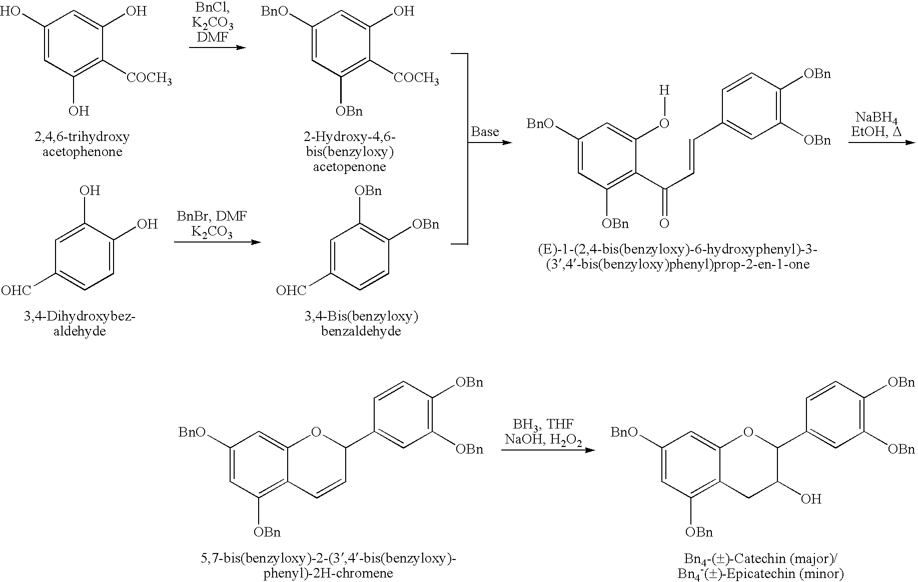

Suitable bases used include piperidine, pyridine, potassium-tert-butoxide and potassium hydroxide in refluxing ethanol, and sodium hydride in N,N-dimethylformamide at about 0° C. Preferably, the cyclizing step (b) is carried out in a mixture of tetrahydrofuran and ethanol using sodium borohydride at about 65° C. Preferably, the oxidizing step is carried out using borane, tetrahydrofuran, hydrogen peroxide, and sodium hydroxide.

The 2-hydroxy-4,6-bis(benzyloxy)-2-acetophenone starting material used in the first step is prepared by benzylating 2,4,6-trihydroxy-acetophenone with a benzyl halide such as benzyl bromide (BnBr) or benzyl chloride (BnCl) in N,N-dimethylformamide (DMF) in the presence of potassium carbonate (K₂CO₃) at room temperature (RT) to about 80° C. The desired compound is isolated after silica gel chromatography and recrystallized from a mixture of dichloromethane and methanol. The 3,4-bis(benzyloxy)benzaldehyde starting material used in the first step is prepared by benzylating 3,4-dihydroxybenzaldehyde with a benzyl halide such as benzyl bromide (BnBr) or benzyl chloride (BnCl) in N,N-dimethylformamide (DMF) in the presence of potassium carbonate (K₂CO₃) at room temperature (RT), preferably using a slight excess of benzyl bromide and potassium carbonate. The preferred amounts are about 2.1 equivalents each. The desired compound is recrystallized from a mixture of ethyl acetate and heptane.

### Part B

### Preparation of a Racemic Mixture of 5,7,3′,4′-Tetra-O-benzyl-(±)-catechin and -(±)-epicatechin from 2-Hydroxy-4,6-bis(benzyloxy)-acetophenone and 5,7-Bis(benzyloxy)-2-(3,4-bis(benzyloxy)phenyl-2H-chromene

The reaction sequence for this process is set out below.

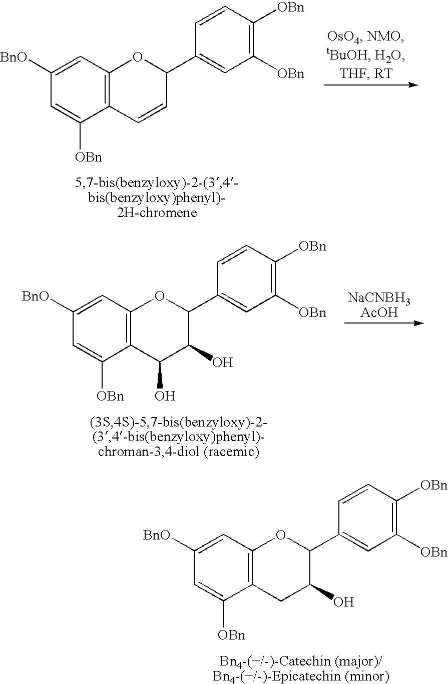

In this process, 5,7-bis(benzyloxy)-2-(3′,4′-bis(benzyloxy)-phenyl-2H-chromene, is dihydroxylated to form racemic (3S,4S)-5,7-bis(benzyloxy)-2-(3′,4′-bis(benzyloxy)phenyl)chroman-3,4-diol, also referred to as 5,7,3,4-tetra-O-benzyl-flavan-3-ene. Preferably, the dihydroxylation is carried out

with osmium tetraoxide (OSO₄) and N-methyl morpholine oxide in a mixture of tert-butanol, water (H₂O), and tetrahydrofuran at room temperature. The desired compound is purified by crystallization using dichloromethane and methyl tert-butyl ether. The compound is reduced to form a racemic mixture of 5,7,3′,4′-tetra-O-benzyl-(±)-catechins and 5,7,3′,4′-tetra-O-benzyl-(O)-epicatechins. Preferably, the reduction is carried out with sodium cyanoborohydride (NaCNBH₃) in acetic acid (AcOH) at 55-60° C. The mixture is chemically resolved to recover the 5,7,3′,4′-tetra-O-benzyl-(+)-catechin or the epimers are separated by chiral preparative high pressure liquid chromatography. The reaction sequence for the second process is set out below.

### Part C

### Preparation of a Racemic Mixture of 5,7,3′,4′-Tetra-O-benzyl-(O)-catechin and -(±)-epicatechin from 3,5-Bis(benzyloxy)-2-(3,4-bis(benzyloxy)-phenol and (E)-3-(3′,4′-bis(benzyloxy)phenyl)prop-2-ene-1-ol

The reaction sequence for this process is set out below.

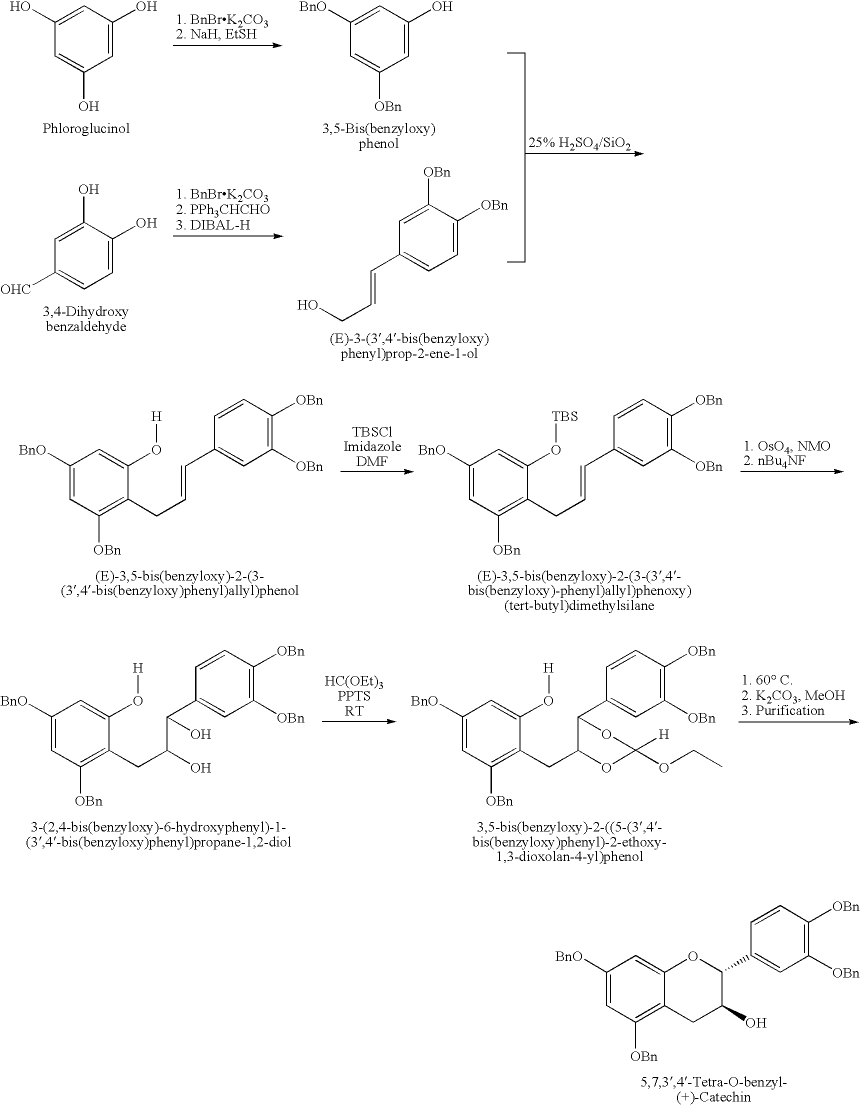

This process comprises the steps of:

(a) coupling 3,5-bis(benzyloxy)phenol with (E)-3-(3′,4′-bis(benzyloxy)-phenyl)prop-2-ene-1-ol under acidic conditions to form (E)-3,5-bis(benzyloxy)-2-(3-(3′,4′-bis(benzyloxy)phenyl)allyl)phenol;

(b) reacting the compound formed in step (a) with tert-butyldimethylsilane chloride and imidazole in dimethylformamide to form (E)-(3,5-bis(benzyloxy)-2-(3-(3′,4′-bis(benzyloxy)phenyl)allyl)phenoxy)(tert-butyl)-dimethysilane;

(c) isolating the compound formed in step (b) by silica gel column chromatography;

(d) dihydroxylating the compound isolated in step (c) osmium tetraoxide and N-methylmorpholine-N-oxide in a mixture of tert-butanol, water, and tetrahydrofuran to form racemic 3-(2,4-bis(benzyloxy)-6-tert-butyldimethylsilyloxy-phenyl-1-(3′,4′-bis(benzyloxy)phenyl)propane-1,2-diol;

(e) removing the tert-butyldimethylsilyl protecting group from the compound of step (d) using tetrabutylammonium fluoride in tetrahydrofuran to form racemic 3-(2,4-bis(benzyloxy)-6-(hydroxyphenyl)-1-(3′,4′-bis(benzyloxy)-phenyl)propane-1,2-diol;

(f) converting the compound formed in step (e) to 3,5-bis(benzyloxy)-2-(5-(3′,4′-bis(benzyloxy)phenyl)-2-ethoxy-1,3-dioxolane-4-yl)phenol; and

(g) converting the compound formed in step (e) to 5,7,3′,4′-tetra-O-benzyl-(+)-catechin by treatment with potassium carbonate in a solvent mixture of methanol and dichloroethane. The reaction mixture is worked up by removing the solvent under vacuum, extracting the residue with ethyl acetate and water, removing the water, drying the ethyl acetate over sodium sulfate, and evaporating the ethyl acetate to recover the crude 5,7,3′,4′-tetra-O-benzyl-(+)-catechin.

In the third step, the phenolic hydroxyl group of (E)-3,4-bis(benzyloxy)-2-(3′,4′-bis(benzyloxy)phenyl)allyl)phenol is protected with a tert-butyldimethylsilyl group introduced by reaction with tert-butyldimethyl chlorosilane at room temperature. When the protected-2H-chromene is treated with 1.5 equivalents of tert-butyldimethyl chlorosilane and 3 equivalents of imidazole in the presence of a catalytic amount of N,N-dimethylaminopyridine and 3 equivalents of triethylamine in dichloromethane at room temperature for 48 hours, the protected compound is isolated in 65-72% yield after silica gel chromatography. When this compound is treated with 1.5 equivalents of imidazole in 15 volumes of N,N-dimethylformamide at room temperature for 24 hours, the protected compound is isolated in only 51% yield. When the amount of the dimethylformamide is reduced to 8.5-10 volumes, the protected compound is obtained in 76-99% yield. Further reducing the amount of N,N-dimethylformamide to 5 volumes results in the isolation of 96% of the protected compound (>99% chemical purity) after silica gel plug purification. On a larger scale, the protected diol is obtained in 81% yield with 98% purity and 81% ee.

In the fifth step, the (1R,2R)- or (1S,2S)-3-(2,4-bis(benzyloxy)-phenylpropane-1,2-diol is deprotected. Removal of the tert-butyldimethylsilyl protecting group is achieved by using n-tetrabutylammonium fluoride and glacial acetic acid at ambient temperature. The crude product obtained after extractive work up is then treated with 25% methyl-tert-butyl ether in ethyl acetate at room temperature to give the desired triol in 80-91% yield and in 88.2% ee as judged by chiral HPLC without the formation of the unknown impurity.

In the sixth step, (1R,2R)- or (1S,2S)-3-bis(benzyloxy)-6-hydroxphenyl-1-(3′,4′-bis(benzyloxy)pehnyl-propane-1,2-diol is cyclized to 5,7,3′,4′-tetra-O-benzyl-(−)-catechin-3-O-propyl ester or 5,7,3′,4′-tetra-O-benzyl-(+)-catechin-3-O-propyl ester upon treatment with triethylorthoformate or preferably triethylorthopropionate and a catalytic amount of pyridinium p-toluenesolfonate via unisolated intermediate 3,5-bis(benzyloxy)-2-((4R,5R)-5-(3′,4′-bis(benzyloxy)phenyl)-2-ethoxy-2-ethyl-1,3-dioxolan-4-yl)phenol in good yield. The reaction, however, produces a number of by-products. When the reaction solvent is changed from 1,2-dichloroethane to dichloromethane, the desired compound is obtained in quantitative yield after extractive work-up. The chloroformate-intermediate 3,5-bis(benzyloxy)-2-(5-(3′,4′-bis(benzyloxy)phenyl)-2-ethoxy-1,3-dioxolan-4-yl)phenol is unstable under normal storage conditions and produces a number of undesired by-products. Hence, the crude product is used in the final step without any further purification. TLC analysis of the crude product shows a minor impurity. The purity, as confirmed by HPLC, was 98% (AUC). Further reaction products obtained from this intermediate produced the desired compound i.e. benzylated catechins but in low yield and purity. A number of other by-products were observed. However, the proprionate-intermediate, 3,5-bis(benzyloxy)-2-((5-(3′,4′-bis(benzyloxy)phenyl)-2-ethoxy-2-ethyl-1,3-dioxolan-4-yl)propyl)phenol is stable and the products obtained from this intermediate were of higher purity as judged by HPLC analysis.

In the final step, the ester group at the 3-hydroxyl position is hydrolyzed, preferably in a mixture of dichloromethane and methanol in the presences of potassium carbonate at room temperature for 24 hours. The use of a mixture of methanol and dichloromethane results in a more rapid reaction. The chiral purity is ˜67% ee as judged by HPLC. Chemical purity is >95%.

### Part D

### Preparation of The Uncommon Epimers 5,7,3′,4′-Tetra-O-Benzyl-(−)-Catechin and -(+)-Epicatechin

The reaction sequence for the process for preparing the uncommon epimers is shown below.

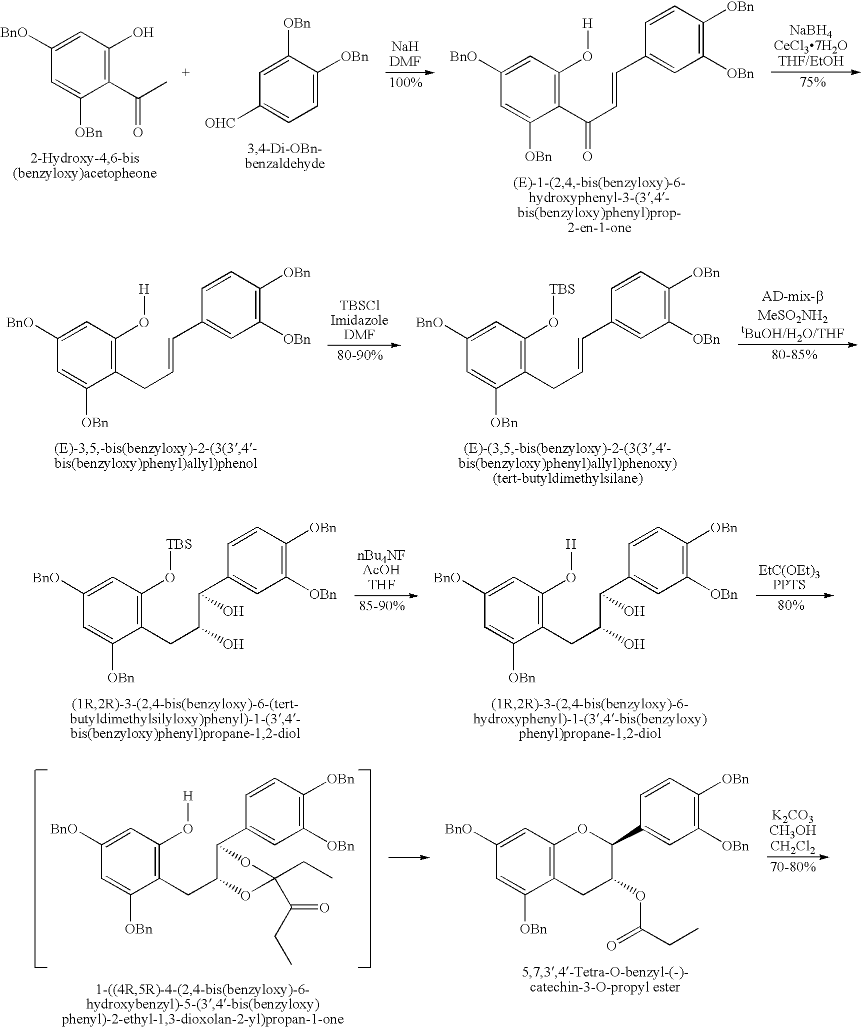

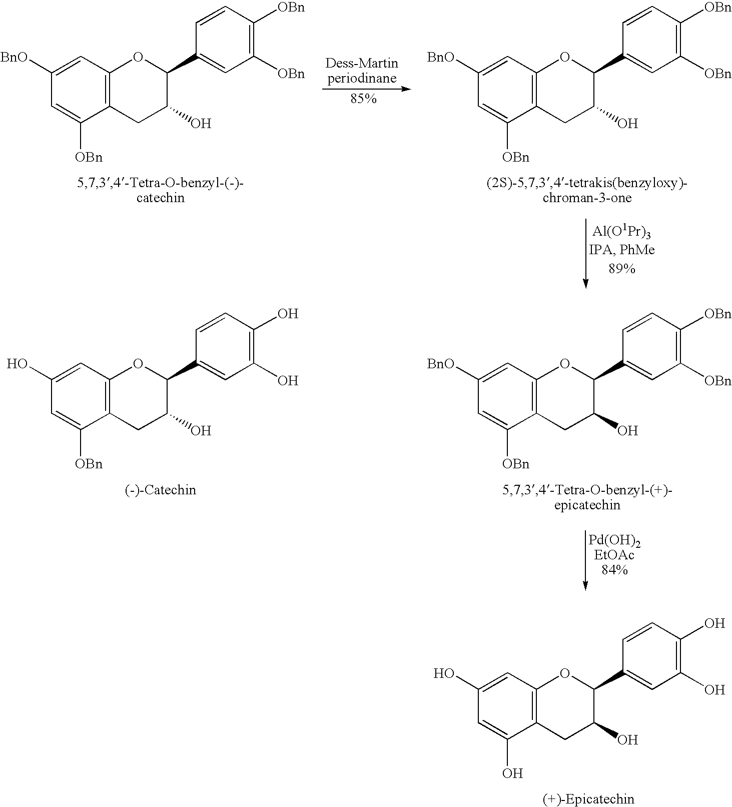

In this seven step process commercially available 2-hydroxy-4,6-bis(benzyloxy)-acetophenone and 3,4-bis(benzyloxy)benzaldehyde are used as the starting materials.

In the first step, (E)-3,5-bis(benzyloxy)-2-(3-(3′,4′-bis(benzyloxy)phenyl)allyl)phenol used in the first step is prepared by condensing 2,4-di-O-benzyl-6-hydroxy-acetophenone with 3,4-bis(benzyloxy)benzaldehyde in the presence of a base, e.g., sodium hydroxide or sodium hydride or potassium hydride or potassium hydroxide, to form (E)-1-(2,4-bis(benzyloxy)-6-hydroxyphenyl-3-(3′,4′-bis(benzyloxy)phenyl)prop-2-en-1-one and cyclizing the resulting compound under reductive conditions.

The selective reduction of the conjugated ketone of (E)-1-(2,4-bis(benzyloxy)-6-hydroxyphenyl-3-(3′,4′-bis(benzyloxy)phenyl)prop-2-en-1-one with sodium borohydride and cerium chloride at 0° C. to 5° C. in a mixture of tetrahydrofuran and ethanol resulted in (E)-3,5-bis(benzyloxy)-2-(3-(3′,4′-bis(benzyloxy)phenyl)allyl)phenol in 76% yield. This is an improved process for the synthesis of this compound.

In the fourth step, the (E)-(3,5-bis(benzyloxy)-2-(3-(3′,4′-bis(benzyloxy)phenyl)allyl)phenoxy)(tert-butyl)dimethylsilane is asymetrically dihydroxylated with AD-Mix-α or AD-Mix-β in the presence of methanesulfonamide in tert-butanol/water using tetrahydrofuran as a co-solvent at ˜0.8° to ˜0.2° C. The use of the tetrahydrofuran as a co-solvent in place of dichloromethane increases the reaction rate (from 96 to 24 hours). Also lower temperatures (˜0.8° to ˜0.2° C. vs. 0 to 5° C.) increase the optical purity of the diol. The desired diol is obtained in good yield with 87-89% ee (as judged by chiral HPLC). The protected diol (1S,2S)-1,2-diol or (1R,2R)-1,2-diol is isolated in quantitative yield after extractive work-up. Similar results should be obtained when AD-mix-α is used.

In the fifth step, the protected diol is treated with 2 equivalents of n-tetrabutylammonium fluoride in tetrahydrofuran (THF) to form (1R,2R)-3-(2,4-bis(benzyloxy)-6-hydroxyphenyl)-1-(3′,4′-bis(benzyloxy)phenyl)propane-1,2-diol which is isolated in quantitative yield. The ee of the compound, however, is only 67% as judged by chiral HPLC. The yield and ee were not consistent. A repeated desilylation gave a yield of 75% and ee of 84%. Attempts to increase the ee of the compound via trituration with hot methyl-tert-butyl ether or various mixtures of ethyl acetate in methyl-tert-butyl ether (80%/20%, 10%/90%, or 75%/25%) result in even lower ee values and the formation of an unidentified impurity. When the deprotection is performed in the presence of an equimolar amount of glacial acetic acid (AcOH) and n-tetrabutylammonium fluoride in tetrahydrofuran at a low temperature (0-5° C.), the stereochemical integrity during the conversion was retained as judged by chiral HPLC. It is believed that the use of acetic acid with the n-tetrabutylammonium fluoride results in the in situ formation of hydrogen fluoride, thus avoiding the basicity which may cause unwanted side reactions. The preferred conditions are the use of equimolar amounts of glacial acetic acid and n-tetrabutylammonium fluoride in tetrahydrofuran at 0-5° C. Tetrahydrofuran could be replaced with dichloromethane.

In the sixth step when pyridinium p-toluenesulfonate (PPTS) is replaced with glacial acetic acid in dichloromethane, the reaction rate is very slow and the desired cyclic orthoformate is obtained in only 26% yield. The formation of the cyclic orthoformate occurs at room temperature under acidic conditions whereas the cyclization occurs at 60°-65° C.

In the last step the dichloromethane solvent cannot be replaced with other solvents such as acetonitrile. The stability of the 3,5-bis(benzyloxy)-2-(5-(3′,4′-bis(benzyloxy)phenyl)-2-ethoxy-1,3-dioxolan-4-yl)phenol is improved by replacing triethylorthoformate with triethylorthopropionate. In the presence of a catalytic amount of pyridinium p toluenesulfonate in 1,2-dichloroethane at 60° C. for about ˜6 hours, the cyclic 3-O propionate ester of 5,7,3′,4′-tetra-O-benzyl-(−)-catechin is formed. The 3-O-propionate ester is more stable than the 3-O-formate ester and is recovered as the sole product (79% yield) after extractive work-up followed by purification by silica gel chromatography.

### Part E

### Purification of Racemic Mixture of 5,7,3′,4′-Tetra-O-Benzyl-(±)-Catechin

A process for preparing enantiomerically pure 5,7,3′,4′-tetra-O-benzyl-(+)-catechin from a racemic mixture comprises the steps of:

(a) esterifying the 3-position of a racemic mixture consisting essentially of 5,7,3′,4′-tetra-O-benzyl-(O)-catechin and 5,7,3′,4′-tetra-O-benzyl-(±)-epicatechin with dibenzoyl-L-tartaric acid monomethyl ester to form a racemic mixture of (O)-(2R,3R)-1-((2R,3S)-5,7-bis(benzyloxy)-2-(3′,4′-bis(benzyloxy)-phenyl)chroman-3-yl)-4-methyl-2,3-bis(benzyloxy)succinate;

(b) fractionally crystallizing the racemic mixture from step (a) to recover enantiomerically pure succinate; and

(c) hydrolyzing the enantiomerically pure succinate from step (b) to form the enantiomerically pure 5,7,3′,4′-tetra-O-benzyl-(+)-catechin.

The dibenzoyl-L-tartaric acid monomethyl ester used in the above process is prepared by an improved process which involves reacting dibenzoyl-L-tartaric acid with methanol in methylene chloride in the presence of 1-hydroxybenztriazole and 1-(3-dimethylaminopropyl)-3-ethylcarbodiimide hydrochloride and working up the reaction mixture.

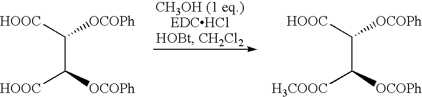

The dibenzoyl-L-tartaric acid monomethyl ester used in the first step of the purification process is prepared by (a) reacting dibenzoyl-L-tartaric acid with methanol in methylene chloride in the presence of 1-hydroxybenztriazole and 1-(3-dimethylaminopropyl)-3-ethylcarbodiimide hydrochloride; and (b) working up the reaction mixture.

The esterifying step is carried out by stirring and then filtering a mixture of N,N-dicyclohexylcarbodiimide in dichloromethane, 5,7,3′,4′-tetra-O-benzyl-(O)-catechin, dibenzoyl-L-tartaric acid monomethyl ester, and 4-dimethylaminopyridine in methylene chloride. The mixture is filtered, concentrated, and purified via silica gel column chromatography. Fractions containing (+)-(2R,3R)-((2R,2S)-5,7-bis(benzyloxy)-2-(3′,4′-bis(benzyloxy)-phenyl))chroman-3-yl-4-methyl)-2,3-bis(benzyloxy)succinate are eluted and the solvent is removed. The combined fractions are dried. The stirring and filtering steps preferably occur under a nitrogen atmosphere, initially at ice bath temperature and then at RT. The (+)-(2R,3R)-((2R,2S)-5,7-bis(benzyloxy)-2-(3′,4′-bis(benzyloxy)phenyl))chroman-3-yl)-4-methyl-2,3-bis(benzyloxy)succinate and the dibenzoyl-L-tartaric acid monomethyl ester are present in a ratio of about 1:1.3 (eq:eq).

The purifying step is preferably carried out using a stationary phase of silica gel mixed with approximately equal volumes of methylene chloride and heptane. A mobile phase of methylene chloride:heptane progresses from a ratio of about 1:1 (v/v) to about 9:1 (v/v).

The fractional crystallization step takes place in a solution of about 80% methylene chloride and about 20% heptane (v/v).

The step of hydrolyzing (+)-(2R,3R)-((2R,2S)-5,7-bis(benzyloxy)-2-(3′,4′-bis(benzyloxy)phenyl))chroman-3-yl)-4-methyl-2,3-bis(benzyloxy)succinate is carried out by dissolving the succinate in potassium hydroxide and methanol, heating at 40-45° C., further diluting with methylene chloride and with potassium hydroxide in methanol. The solution is heated for about 4 h. The solvent is removed in vacuo. The recovered product is suspended in water, heated, and then concentrated in vacuo. The reaction is diluted with methylene chloride, washed, dried over sodium sulfate and filtered. The solvent is removed in vacuo and the crude product is purified by silica gel chromatography using methylene chloride in heptane. The fractions containing the 5,7,3′,4′-tetra-O-benzyl-(+)-catechin are combined and the solvent is removed. The resulting crystalline product is the enantiomerically pure 5,7,3′,4′-tetra-O-benzyl-(+)-catechin.

While the above hydrolysis in potassium hydroxide produced the desired product, i.e., 5,7,3′,4′-tetra-O-benzyl-(+)-catechin, in good yield, other bases such as lithium hydroxide in tetrahydrofuran, or sodium hydroxide, or milder bases may also be used: There is a possibility for transesterification to occur if lower order alcohols are used as the solvent.

In the examples which follow all parts are by weight unless indicated otherwise, eq is equivalent, m is mole(s), v is volume, RT is room temperature, h is hour(s), min is minutes), HPLC is high pressure liquid chromatography where the results are reported as AUC % (area percent under the curve) at a wavelength of 280 nm.

The following reversed phase chiral HPLC procedure was developed to determine the chiral purity of 5,7,3′,4′-tetra-O-benzyl-(O)-catechin and 5,7,3′,4′-tetra-O-benzyl-(±)-epicatechin. All reagents were HPLC grade. The racemate reference materials were obtained from internal sources. A standard HPLC system with PDA detection and data system was used. The stationary phase consisted of a Chiralpack AD-RH analytical column (Chiral Technologies, Inc., West Chester, Pa.), with an I.D. of 150×4.6 mm and a particle size of 5μ. The binary mobile phase consisted of an (A) phase of water and a (B) phase of acetonitrile. Reference material, to be used for peak identification, is prepared for HPLC analysis by placing about 2-3 mg of the reference material into an HPLC vial, dissolving it in 1 mL of acetonitrile, and vortexing the solution to achieve complete dissolution. Samples are prepared for HPLC analysis by placing about 2-3 mg of sample into an HPLC vial, dissolving it in 1 mL of acetonitrile, and vortexing the solution to achieve complete dissolution. HPLC is effected at a column temperature of 60° C. and a flow rate of 1.0 mL/min, in a binary mobile phase of isocratic A:B ratio of 35:65. Run time is 40 minutes, with 1 minute for equilibration. Sample size (injection volume) is 5 μ/L. Detection wavelength is 280 nm, and peak width (response time) is >0.1 min. The injection format consists of at least one blank, followed by one sample, which is followed by one reference material sample if needed for peak identification. The suitability of the above system for determining chiral purity of the four 5,7,3′,4′-tetra-O-benzyl(O)-catechins and (±)-epicatechins is shown by the relative retention times and tailing factors of the four epimers, as set forth below:

| | Retention | Theoretical | |
|---|---|---|---|
| Compound | time (min) | Plate | Tailing Factor |
| 5,7,3′,4′-tetra-O-benzyl-(+)- | 13.15 | 4516 | 1.11 |
| catechin Bn₄-(+)-C | | | |
| 5,7,3′,4′-tetra-O-benzyl-(−)- | 14.98 | 4285 | 1.12 |
| catechin Bn₄-(−)-C | | | |
| 5,7,3′,4′-tetra-O-benzyl-(−)- | 16.45 | 4307 | 1.17 |
| epicatechin Bn₄-(−)-EC | | | |
| 5,7,3′,4′-tetra-O-benzyl-(+)- | 24.58 | 3315 | 1.25 |
| epicatechin Bn₄-(+)-EC | | | |

The theoretical plate refers to the ability of the HPLC column to keep the sample bands narrow. Columns with large plate numbers give narrow bands; long columns packed with small particles give the highest plate numbers. Tailing factor refers to the asymmetrical shape of a peak, technically defined as having an asymmetry factor>1.

Chiral purity of each epimer in a sample is calculated as follows:

% chiral purity of Bn₄-(+)-C═Bn₄-(+)-C/total peak areas of Bn₄-(+)-C and Bn₄-(−)-C.

% chiral purity of Bn₄-(−)-C═Bn₄-(−)-C/total peak areas of Bn₄-(+)-C and Bn₄-(−)-C.

% chiral purity of Bn₄-(+)-EC=Bn₄-(+)-EC/total peak areas of Bn₄-(+)EC and Bn₄-(−)EC.

% chiral purity of Bn₄-(−)-EC=Bn₄-(−)-EC//total peak areas of Bn₄-(+)-EC and Bn₄-(−)-EC.

Following is a typical chiral HPLC chromatogram separating a mixture of Bn₄-(+)-C, Bn₄-(−)-C, Bn₄-(−)-EC and Bn₄-(+)-EC:

Using the above developed chiral HPLC method, the four isomers namely Bn₄-(+)-C, Bn₄-(−)-C, Bn₄-(−)-EC and Bn₄-(+)-EC could be separated.

The following analytical procedures were used:

Chemical Purity

Chemical purity was determined using a standard HPLC system with PDA detection and data system. The column was Agilent, Zorbax, 3.5 μm, SB-C8, 2.1×50 mm column (Cat#871700-906). The column temperature was 25° C. The column was equilibrated for 2 minutes before use. The mobile phases were A: 0.01% trifluoroacetic acid in water: add 100 μL trifluoroacetic acid into 1 L of water, and mix well and B: 0.01% trifluoroacetic acid in acetonitrile: Add 100 μL trifluoroacetic acid into 1 L of acetonitrile and mix well. The flow rate was 0.8 ml/min. The detection wavelength was 280 nm. The injection volume was 5 μL. The gradient program was:

| Time (Min.) | % B |
|---|---|
| 0 | 5 |
| 4 | 100 |
| 6 | 100 |

Optical Purity

The optical purities of (1R,2R)-3-(2,4-bis(benzyloxy)-6-(tert-butyl-dimethylsilyloxy)-phenyl-1-(3′,4′-bis(benzyloxy)phenyl)propane-1,2-diol and (1R,2R)-3-(2,4-bis-(benzyloxy)-6-hydroxyphenyl)-1-(3′,4′-bis(benzyloxy)-phenyl)propane-1,2-diol were determined by a standard HPLC system with PDA detection and data system. The column was Chiralcel OJ-RH, 5μ, 150×4.6 mm analytical column (Cat. #17724 (Chiral Technologies, Inc.)). The column temperature was 40° C. The mobile phase was Isocratic A (water)/B (ACN) Acetonitrile (35/65 v/v). The flow rate was 1 mL/min. The run time was 30 min. The detection wavelength was 210 nm. The injection volume was 5 μL.

### EXAMPLES

### Example 1

### Preparation of 2-Hydroxy-4,6-bis(benzyloxy)-acetophenone

This example describes the preparation and purification of the title compound from commercially available 2,4,6-trihydroxy acetophenone. A stirred suspension of 2,4,6-trihydroxyacetophenone (10 g, 0.054 mol, 1 eq) and potassium carbonate (16.3 g, 0.118 mol, 2.2 eq) in N,N-dimethylformamide (100 mL, 10 vol, 1 g/10 mL) was heated at 80° C. To this suspension was added benzyl chloride (13.6 mL, 0.118 mol, 2.2 eq) in one portion. The suspension was kept at 80° C. for about 1 h. The reaction mixture was cooled to RT and carefully acidified with 1 M hydrochloric acid (200 mL). The aqueous layer was extracted twice with ethyl acetate (100 mL) The combined organic layers were washed twice with water (100 mL) and twice with brine (100 mL), dried over sodium sulfate, and filtered. The solvent was removed under vacuum to afford a red viscous oil. The oil was dissolved in dichloromethane and passed through a 200 g plug of silica gel. The silica gel was eluted with 1 L of dichloromethane. The combined solvent was evaporated under reduced pressure to produce an oil which solidified upon standing at RT. The yield was 18.7 g. HPLC purity was 69% purity. The product contained 19.7% of a tribenzyl impurity.

The crude solid was dissolved in hot dichloromethane (15 mL) and methanol (20 mL) was added slowly. The solids started to appear immediately. The suspension was allowed to cool to RT with agitation. The solids were suction filtered, washed with methanol (75 mL), and dried under high vacuum to produce 9.1 g of an off-white solid. The yield was 49%. HPLC purity was 96.9%. The product contained about 2.54% of the tribenzyl impurity.

A number of reaction conditions and various benzylating reagents (benzyl bromide (BnBr) and benzyl chloride (BnCl) were screened to optimize the selective benzylation. The results are set out in Table 1.

TABLE 1

| Amount of | | | | | | |
|---|---|---|---|---|---|---|
| Starting | Potassium | Benzyl | Dimethyl- | | | |
| material | Carbonate | Halide | formamide | Conditions | Results | Yield |
| 1 g | 1.73 g | BnBr | 8 mL | stirred at | isolated after | 38.5% |
| 5.9 mol | 12.5 mol, | 2 eq | | RT for | silica get | |
| 1 eq | 2.1 eq | | | 21 h | chromatography | |
| 0.3 g | 2.1 eq | BnBr | 8 mL | stirred at | Isolated after | 38.5% |
| 1 eq | | 2 eq | | RT for | silica get | |
| | | | | 24 h | chromatography | |
| 0.3 g | 1.73 g | BnCl | 10 mL | stirred at | desired product | 58.6% |
| 1 eq | 12.5 mol | 2.2 eq | | RT then at | | |
| | 2.1 eq | | | 40° C. | | |
| 5 g | 2.2 eq, | BnCl | 10 mL | stirred at | desired product | 46.6% |
| 1 eq | | 2.2 eq | | 40° C. for 6 h | | |
| | | | | then at | | |
| | | | | 80° C. for | | |
| | | | | 1 h | | |
| 0.3 g | 2.2 eq | BnCl | 10 mL | kept at | desired product | 76.6% |
| 1 eq | | 2.2 eq | | 80° C. for | | |
| | | | | 1 h | | |
| 0.3 g | 2.2 eq | BnCl | 10 mL | kept at | desired product | 68.5% |
| 1 eq | | 2.2 eq | | 60° C. for | | |
| | | | | 1 h | | |
| 0.3 g | 2.2 eq | BnCl | 10 mL | kept at | desired product | 74.5% |
| 1 eq | | 3 eq | | 60° C. for | | |
| | | | | 1 h | | |
| 0.3 g | 2.1 eq | BnCl | 10 mL | kept at | desired product | 70.3% |
| 1 eq | | 2.1 eq | | 60° C. | | |
| 0.3 g | 3 eq | BnCl | 10 mL | kept at | desired product | 70.8% |
| 1 eq | | 3 eq | | 40° C. for | | |
| | | | | 1 h | | |
| 14.7 g | 2.2 eq | BnCl | 150 mL | kept at | crude product | |
| 1 eq | | 2.2 eq | | 80° C. for | | |
| | | | | 1 h | | |

### Example 2

### Preparation of 3,4-Bis(benzyloxy)benzaldehyde

To a stirred suspension of commercially available 3,4-dihydroxybenzaldehyde (68 g, 0.492 mol, 1 eq), potassium carbonate (170 g, 1.23 mol, 2.5 eq) in N,N-dimethylformamide (400 mL, ˜5.9 vol., ˜5.9 mL/g) was added slowly benzyl bromide (185.14 g, 1.08 mol, 2.2 eq) at RT with agitation under a nitrogen atmosphere. An exotherm was observed during the benzyl bromide addition as the internal temperature rose from 18.8° to 35.4° C. Completion of the reaction was monitored by TLC. The reaction mixture was diluted with 200 mL of water and 125 mL of 50% aqueous hydrochloric acid. The reaction mixture was extracted with 500 mL of ethyl acetate and then with 200 mL of ethyl acetate. The combined organic layers were washed with 500 mL of water and 500 mL of a brine solution, dried over 200 g of sodium sulfate, and filtered. The solvent was removed in vacuum to give a beige-colored semi-solid which was dissolved in 150 mL of hot ethyl acetate. Then, 600 mL of heptane was slowly added with agitation. The mixture was cooled to RT and allowed to stir overnight. The solids were suction filtered and then washed twice with 200 mL of a mixture (v/v) of 10% ethyl acetate and 90% heptane and dried under high vacuum. The yield was 138.2 g (88.2%). HPLC purity was 100%.

### Example 3

### Preparation of (E)-1-(2,4-bis(benzyloxy)-6-hydroxyphenyl-3(3′,4′-bis(benzyloxy)phenyl)prop-2-en-1-one

To an ice cold suspension of sodium hydride (60% dispersion in oil, 1.2 g, 0.0286 mol, 1.3 eq) and 2,4-bis(benzyloxy)-6-hydroxy-acetophenone (7.66 g, 0.022 mol, 1 eq) in N,N-dimethylformamide (130 mL) under a nitrogen atmosphere was added 3,4-bis(benzyloxy)benzaldehyde (7 g, 0.022 mol, 1 eq) in dimethylformamide (30 mL) slowly over a period of 5 min. The resulting solution was stirred for 5 min at ice bath temperature and then at RT for about 1.5 h. Consumption of the starting material was monitored by TLC. The reaction mixture was diluted with dichloromethane chloride (200 mL) and washed with 0.3 N hydrochloric acid (300 mL), water (250 mL), saturated aqueous sodium bicarbonate (150 mL), and brine (150 mL), dried over sodium sulfate, and filtered. The solvent was removed under vacuum to give a semi-solid product. The crude product was treated with hot methanol (250 mL) for about 0.5 h and then cooled to RT. The resulting solids were suction filtered, washed twice with methanol (15 mL), and dried under high vacuum at RT for about 18 h. The yield was 12.2 g (85.5%).

### Example 4

### Preparation of 5,7-Bis(benzyloxy)-2-(3′,4′-bis(benzyloxy)-phenyl-2H-chromene

To a stirred solution of (E)-1-(2,4-bis(benzyloxy)-6-hydroxyphenyl-3-(3′,4′-bis(benzyloxy)phenyl)prop-2-en-1-one (20 g, 0.0308 mol, 1 eq) in tetrahydrofuran (400 mL) and ethanol (200 mL) was added sodium borohydride (1.4 g, 0.037 mol) at RT with stirring. The resulting reaction mixture was slowly heated at reflux. After a 4-5 h reflux, HPLC analysis of the reaction mixture indicated consumption of the starting material and the presence of a new peak. The reaction mixture was cooled to RT and diluted with dichloromethane (300 mL). The reaction mixture was washed with water (100 mL), saturated aqueous sodium bicarbonate (100 mL), and brine (100 mL), dried over sodium sulfate (50 g), and suction filtered. The filtrate containing the desired compound was used in the next example without purification.

If the (E)-1-(2,4-bis(benzyloxy)-6-hydroxyphenyl-3-(3′,4′-bis(benzyloxy) needs to be isolated, it can be accomplished by silica gel chromatography using about 20 to about 40% dichloromethane in heptane (v/v) as an eluant.

### Example 5

### Preparation of 5,7,3′,4′-Tetra-O-Benzyl-(±)-Epicatechin

The 3,4-double bond of (E)-1-(2,4-bis(benzyloxy)-6-hydroxyphenyl-3-(3′,4′-bis(benzyloxy)phenyl)prop-2-en-1-one was oxidized under oxidative hydroboration conditions by treating the compound first with borane-tetrahydrofuran at 0° to −5° C. for 4 h and at RT for 2 h, followed by removing the solvent under pressure and treating the residue with 1 M sodium hydroxide and 30% hydrogen peroxide solutions at RT for 2 h. The reaction mixture was diluted with methylene chloride and washed with aqueous potassium carbonate, water, and a brine solution. The organic layer was dried over sodium sulfate, filtered, and the solvent was removed under pressure. The crude product was purified by silica gel chromatography to afford 5,7,3′,4′-tetra-O-benzyl-(±)-catechin. The racemic mixture was isolated as an off-white solid. ¹H NMR analysis indicated that 5,7,3′,4′-tetra-O-benzyl-(±)-catechin was the major product. Chiral HPLC analysis of the product indicated that it was a mixture of 5,7,3′,4′-tetra-O-benzyl-(±)-catechin and 5,7,3′,4′-tetra-O-benzyl-(±)-epicatechin. Minor amounts of 5,7,3′,4′-tetra-O-benzyl-(−)-epicatechin and 5,7,3′,4′-tetra-O-benzyl-(+)-epicatechin were present.

### Example 6

### Preparation of Racemic (3S,4S)-5,7-Bis(benzyloxy)-2-(3′,4′-bis(benzyloxy)phenyl)chroman-3,4-diol

To a stirred solution of tert-butanol (20 mL), tetrahydrofuran (25 mL), 3% aqueous osmium tetraoxide solution (0.52 mL), and 50% aqueous N-methyl morpholine N-oxide solution (0.8 mL) was added a solution of 5,7-bis(benzyloxy)-2-(3′,4′-bis(benzyloxy)phenyl-2H-chromene (obtained from (E)-1-(2,4-bis(benzyloxy)-6-hydroxyphenyl-3-(3′,4′-bis(benzyloxy)phenyl)prop-2-en-1-one in solution) in tetrahydrofuran (15 mL). The resulting solution was stirred at RT for about 1.5 h. Completion of the reaction was monitored by HPLC analysis. The reaction mixture was diluted with methylene chloride (80 mL) and washed with 5% aqueous sodium sulfate (30 mL), water (30 mL), saturated aqueous sodium bicarbonate (30 mL), and a brine solution (30 mL). The organic layer was dried over sodium sulfate and filtered. The solvent was removed under vacuum to produce an off-white solid. The solid was dissolved in methylene chloride (10 mL) and methyl tert-butyl ether (20 mL) was added. The mixture was stirred at 50° C. for about 10 min. The mixture was cooled to RT and the solids were suctions filtered, washed three times with methyl tert-butyl ether (5 mL), and dried under high vacuum at 50° C. for 1 h. The yield was 1 g (48%).

### Example 7

### Preparation of Racemic Mixture of 5,7,3′,4′-Tetra-O-benzyl-(+)-catechin and 5,7,3′,4′-Tetra-O-benzyl-(±)-epicatechin

The racemic mixture of (3S,4S)-5,7-bis(benzyloxy)-2-(3′,4′-bis(benzyloxy)phenyl)chroman-3,4-diols from Example 6 was reduced using sodium cyanoborohydride in glacial acetic acid. The mixture was heated at about 50°-55° C. for 1 h. TLC analysis indicated that the starting diol material was consumed. The reaction mixture was concentrated under pressure to dryness and diluted with methylene chloride and washed with aqueous sodium hydroxide, water, and brine solution. The organic layer was concentrated and chased with toluene, dissolved in methylene chloride, and purified by silica gel chromatography. The crude product obtained was further purified by silica gel chromatography. The yield was 82%. HPLC analysis indicated that the product was a mixture of 5,7,3′,4′-tetra-O-benzyl-(±)-catechin and 5,7,3′,4′-tetra-O-benzyl-(O)-epicatechin in a ratio of 87.5:11.5 (% AUC).

### Example 8

### Preparation of Enantiomerically Pure 5,7,3′,4′-Tetra-O-benzyl-(+)-Catechin

### Part A—Preparation of Dibenzoyl-L-Tartaric Acid Monomethyl Ester.

A total 1.9 g (10 mol, 1 eq) of 1-(3-dimethylaminopropyl)-3-ethylcarbodiimide was added to a stirred solution of dibenzoyl-L-tartaric acid (3.58 g, 10 mol, 1 eq) and 1-hydroxybenzotriazole hydrate (1.35 g, 10 mol, 1 eq) in dichloromethane (200 mL) at RT with stirring. The resulting suspension was stirred at RT for 10 min. Methanol (0.4 mL, 10 mol, 1 eq) was added slowly over a period of ˜2 min. The resulting suspension was stirred at RT for 2 h. Consumption of the starting material was monitored by TLC. The reaction mixture was diluted with dichloromethane (50 mL) and washed twice with water (50 mL). The organic layer was dried with sodium sulfate and filtered through a plug of silica gel. The silica gel plug was washed twice with methylene chloride (100 mL) and then washed three times with ethyl acetate (50 mL). Fractions containing the desired product (as judged by TLC) were combined. The solvent was removed by vacuum. The product was an off-white solid. The yield was 88%.

### Part B—Esterification of Racemic 5,7,3′,4′-Tetra-O-Benzylated (O)—Catechin.

A total of 30 mL of 1 M N,N-dicyclohexylcarbodiimide in methylene chloride was slowly added to an ice-cold mixture of 5,7,3′,4′-tetra-O-benzyl-(±)-catechin (1.9 g, 0.0029 mol, 1 eq) dibenzoyl-L-tartaric acid monomethyl ester (1.45 g, 0.0038 mol, 1.3 eq) from Part A, 4-dimethylaminopyridine (50 mg) in methylene chloride under a nitrogen atmosphere. The mixture was stirred at ice bath temperature for 4 min and then at RT for approximately 20 min as the progress of the reaction was monitored by TLC and HPLC. The reaction mixture was suction filtered to remove N,N′-dicyclohexylurea. The filtrate was concentrated under vacuum to a volume of approximately 5 mL and loaded on a silica gel column (36 g) in dichloromethane. The product was purified by silica gel column chromatography using dichloromethane: heptane (1:1 to 9:1, v/v). The fractions containing the desired product were combined. The solvent was removed under vacuum. The combined fractions were further dried under high vacuum at RT to produce the desired product as an off-white solid. The yield was 98%. ¹H NMR analysis indicated that the mixture contained all four esterified, benzyl-protected catechin and epicatechin epimers.

Part C—Fractional Crystallization of (+)-(2R,3R)-((2R,3S)-5,7-Bis(benzyloxy)-2-(3′,4′-bis(benzyloxy)phenyl)chroman-3-yl)-4-methyl-2,3-bis(benzyloxy)succinate.

The mixture from Part B was further recrystallized from hot dichloromethane:heptane (8:2, v/v, ˜1 g/2 mL) at 50° C. twice followed by three crystallizations with dichloromethane:heptane (8:2, v/v, 1 g/3 mL) to produce the enantiomerically pure (+)-(2R,3R)-((2R,3S)-5,7-bis(benzyloxy)-2-(3′,4′-bis(benzyloxy)phenyl)chroman-3-yl)-4-methyl-2,3-bis(benzyloxy)succinate as an off-white solid. Progress of the crystallization was monitored by ¹H NMR after each crystallization. The yield was 62%.

### Part D—Preparation of Enantiomerically Pure 5,7,3′,4′-Tetra-O-Benzyl-(+)-Catechin.

A solution of the enantiomerically pure compound from Part C (3.2 g, 1 eq) in 0.05M potassium hydroxide in methanol (200 mL) was heated at 40°-45° C. The resulting thick gel was further diluted with dichloromethane (30 mL) and 0.05M potassium hydroxide in methanol (225 mL) and heated at 40°-45° C. for approximately 4 h. The solvent was removed under vacuum. The solid was suspended in water (˜200 mL), heated at 700-74° C. (bath temperature) for 1 h, and concentrated under vacuum for approximately 10 min. The concentrated reaction mixture was diluted with dichloromethane (100 mL), washed once with water (20 mL), washed twice with brine (50 mL), dried over sodium sulfate, and filtered. The solvent was removed under vacuum. The crude product was purified by silica gel chromatography using 50-100% dichloromethane in heptane. Fractions containing the desired product were combined and the solvent was removed in vacuo. The product was crystallized by dissolving it in methylene chloride (10 mL) and methyl tert-butyl ether (75 mL) and heating to 70°-75° C. Hexane (75 mL) was slowly added to the hot solution until a slightly cloudy solution appeared. Approximately 45 mL of distillate was collected using a Dean-Stark apparatus. The solution was allowed to cool to RT with agitation. The solid was suction filtered, washed with methyl tert-butyl ether and dried under high vacuum to give the desired epimer as an off-white solid. The yield was 73%. HPLC purity was 100%. Chiral HPLC analysis showed 97.93% of 5,7,3′,4′-tetra-O-benzyl-(+)-catechin and 2.07% of 5,7,3′,4′-tetra-O-benzyl-(−)-catechin. Optical purity was 96% ee.

### Example 9

### Synthesis of (E)-3,5-Bis(benzyloxy)-2-(3-(3′,4′-bis(benzyloxy)phenyl)allyl)phenol

(E)-(3,5-Bis(benzyloxy)-2-(3-(3′,4′-bis(benzyloxy)phenyl)-allyl)phenol was prepared by coupling 3,5-bis(benzyloxy)phenol and (E)-3-(3′,4′-bis(benzyloxy)phenyl-prop-2-ene-1-ol under acidic conditions using 25% sulfuric acid/silica gel. See L. Li et al., Org. Letts. 2001, 3(5), 739. The desired product was isolated after silica gel column chromatography. The yield was 35-40%.

### Example 10

### Improved Process for the Preparation of (E)-3,5-bis(benzyloxy)-2-(3-(3′,4′-bis(benzyloxy)-phenyl)ally)phenol

To a solution of ethanol (236 mL) and tetrahydrofuran (800 mL) was added cerium chloride heptahydrate (74 g, 198.0 mmol, 2.5 eq) at RT. The mixture was stirred at RT until a clear solution was obtained. To this was added (E)-1-(2,4-bis(benzyloxy)-6-hydroxyphenyl)-3-(3′,4′-bis(benzyloxy)phenyl)prop-2-en-1-one (51.4 g, 79.23 mmol, 1 eq) followed by tetrahydrofuran (500 mL). The solution was stirred at RT for -10 min and then cooled to −1.5° to −0.2° C. (internal temperature) with agitation. Solid sodium borohydride (7.5 g, 197.37 mmol, 2.5 eq) was added in portions while keeping the internal temperature ≦−0.3° C. throughout the addition. It took ˜0.5 h for the addition of sodium borohydride on this scale. The mixture was stirred at this temperature (−0.8° to −0.3° C.) for ˜2.5 h. Completion of the reaction was monitored by HPLC. The reaction mixture was quenched with 5% aqueous citric acid (167 mL) followed by ethyl acetate (1.5 L). The mixture was stirred as the internal temperature rose to −12° C. The organic layer was separated and washed with water (2×1 L, 1×800 mL) and brine (1×500 mL), dried sodium sulfate, and filtered. The solvent was removed in vacuo to give a semi-solid. HPLC analysis indicated 86% of the desired product and 14% of a by-product (AUC). The crude product was purified by silica gel chromatography using heptane/dichloromethane/ethylacetate (25/25/0.5, v/v/v) to give the desired compound as an-off white solid. The yield was 38 g, (75.5%). HPLC purity was 99.5% (AUC).

¹H NMR (300 MHz, CDCl₃) δ=3.55 (d, J=5.4 Hz, 2H), 4.94-5.08 (m, 5H), 5.12 (d, J=4.4 Hz, 4H), 6.04-6.2 (m, 2H), 6.22-6.4 (m, 2H), 6.82 (s, 2H), 6.97 (d, J=1.2 Hz, 1H), 7.18-7.5 (m, 20H).

¹³C NMR (75 MHz, CDCl₃) δ=26.41, 70.19, 70.43, 71.46, 93.63, 95.26, 107.03, 112.78, 115.3, 119.91, 126.7, 127.29, 127.33, 127.41, 127.53, 127.76, 127.79, 127.85, 128.02, 128.45, 128.46, 128.53, 128.62, 130.18, 136.45, 137.13, 137.24, 146.46, 148.19, 155.18, 157.93, 158.86

### Example 11

### Preparation of (E)-(3,5-Bis(benzyloxy)-2-(3-(3′,4′-bis-(benzyloxy)phenyl)allyl)phenoxy)(tert-butyl)dimethylsilane

The reaction between (E)-3-(3,5-bis(benzyloxy)-2-(3′,4′-bis(benzyloxy)-phenyl)allyl)phenol and tert-butyldimethylchlorosilane was performed in N,N-dimethylformamide in the presence of imidazole at RT (Org. Letts. 2001 3(5), 739). The desired product was isolated after silica gel column chromatography.

### Example 12

### Preparation of (E)-(3,5-Bis(benzyloxy)-2-(3-(3′,4′-bis(benzyloxy)-phenyl)allyl)phenoxy)(tert-butyl)dimethylsilane

To a solution of (E)-3-(3,5-bis(benzyloxy-2-(3′,4′-bis(benzyloxy)-phenyl)allyl)phenol (95 g, 150 mmol, 1 eq) in dimethylformamide (450 mL, 4.7 vol) was added imidazole (30.63 g, 450 mol, 3 eq) with stirring at RT. To this, tert-butyldimethylchlorosilane (45.17 g, 300 mmole, 2 eq) was added in portions. The resulting reaction mixture was stirred at RT for 16 h. TLC indicated completion of the reaction. The reaction mixture was poured onto a mixture of ice-water (500 g) and extracted with ethyl acetate (1×500 mL, 1×250 mL). The organic layers were combined, dried with sodium sulfate, and filtered. The solvent was removed in vacuo to give the crude product as an oil. The crude product was purified by passage through a silica gel plug (˜33% loading) using 15% ethyl acetate in heptane (v/v) to give the desired compound as an oil. The yield was 95 g. HPLC purity was 100% (AUC).

¹H NMR (300 MHz, CDCl₃) δ=0.1 (s, 6H), 0.92 (s, 9H), 3.46 (d, J=5.7 Hz, 2H), 4.88 (d, J=3.5 Hz, 4H), 4.98 (d, J=3.5 Hz, 4H), 5.92-6.22 (m, 4H), 6.58 6.74 (m, 2H), 6.8 (s, 1H), 7.1-7.4 (m, 20H)

¹³C NMR (75 MHz, CDCl₃) δ=−4.08, 18.23, 25.65, 25.84, 26.84, 70.15, 70.18, 71.36, 71.48, 93.95, 98.53, 112.33, 112.68, 115.33, 119.58, 127.25, 127.31, 127.35, 127.68, 127.7, 127.87, 127.92, 128.39, 128.41, 128.44, 128.59, 129.03, 137.31, 137.43, 137.46, 149.08, 158.09, 158.35.

MS=749.4 [M⁺+H]

### Example 13

### Preparation of (1R,2R)-3-(2,4-bis(benzyloxy)-6-(tert-butyl dimethylsilyloxy)phenyl)-1-(3′,4′-bis(benzyloxy)phenyl)propane-1,2-diol

To a cold solution (0-2° C.) of AD-mix-β (450 g) in a mixture of tert-butanol and water (1.2 L) was added a cold solution of (E)-(3,5-bis(benzyloxy)-2-(3-(3′,4′-bis(benzyloxy)phenyl)allyl)phenoxy)(tert-butyl)dimethysilane (93 g, 124.3 mmol, 1 eq) in tetrahydrafuran (1.2 L) followed by the addition of methanesulfonamide (15.18 g, 159.8 mmol, 1.26 eq). The resulting mixture was then stirred for 28 h, while keeping the internal temperature at between 0°-2° C. TLC indicated completion of the reaction. Sodium meta bisulfite solution (10% aqueous, w/v, 2 L) was added and the mixture was allowed to warm to RT. The reaction mixture was extracted with ethyl acetate (1×4 L). The organic layer was dried over sodium sulfate and filtered. The solvent was removed in vacuo to give the crude product. The crude product was purified by passage through a silica gel plug (20% loading) to give the desired compound. The yield was 77.43 g (80%). HPLC purity was 96.3% (AUC). Chiral HPLC was 86% ee.

¹H NMR (300 MHz, CDCl₃) δ=0.01 (s, 6H), 0.74 (s, 9H), 2.4 (d, J=5.6 Hz, 1H), 2.64 (d, J=7.2 Hz, 2H), 3.0 (d, J=3.2 Hz, 1H), 3.58-3.72 (m, 1H), 4.22 (q, J=3.3 Hz, 1H), 4.8 (d, J=4.3 Hz, 1H), 4.91 (s, 1H), 4.94 (s, 1H), 5.92 (d, J=2.2 Hz, 1H), 6.1 (d, J=2.2 Hz, 1H), 6.63 (s, 2H), 6.83 (s, 1H), 7.04-7.3 (m, 20H).

¹³C NMR (75 MHz, CDCl₃) δ=−4.2, −4.02, 18.24, 25.82, 27.65, 70.24, 70.52, 71.31, 71.47, 75.57, 76.6, 94.26, 98.91, 109.92, 113.81, 115.15, 119.86, 127.23, 127.29, 127.33, 127.49, 127.7, 128.03, 128.08, 128.38, 128.42, 128.66, 128.71, 134.68, 136.52, 136.9, 137.4, 155.25, 158.36, 158.43.

[α]²⁰_{D}=+0.28° (c=1, CH₂Cl₂)

### Example 14

### Preparation of (1R,2R)-3-(2,4-bis(benzyloxy)-6-hydroxy phenyl)-1-(3′,4′-bis(benzyloxy)phenyl)propane-1,2-diol

To a cold (0°-5° C.) solution of (1R,2R)-3-(2,4-bis(benzyloxy)-6-tert-butyldimethylsilyloxyphenyl)-1-(3′,4′-bis(benzyloxy)phenyl)propane-1,2-diol (54 g, 68.96 mmol, 1 eq) and glacial acetic acid (7.82 mL, 137.93 mmol, 2 eq) in tetrahydrofuran (600 mL) was slowly added a solution of n-tetrabutylammonium fluoride (1 M solution in tetrahydrofuran, 137.93 mL, 137.93 mmol, 2 eq) over a period of 1 h. The reaction mixture was allowed to stir at ice bath temperature for 2 to 3 h, until TLC (ethyl acetate/heptane, 1/1, v/v) indicated completion of the reaction. The tetrahydrofuran was removed in vacuo and the reaction mixture was quenched with a cold solution of 5% aqueous sodium bicarbonate and extracted with dichloromethane (2×400 mL). The combined organic layer was passed through a silica gel plug (260 g) using dichloromethane (500 mL). The filtrates were combined and the solvent was removed in vacuo to give the desired product as an off-white solid. The yield was 41.9 g (91%). HPLC purity was 98.1% (AUC).

¹H NMR (300 MHz, CDCl₃) δ=2.4-2.6 (m, 2H), 2.68-2.8 (m, 1H), 3.7-3.9 (m, 1H), 4.3 (t, J=4.8 Hz, 1H), 4.8-5.22 (m, 11H), 6.1 (d, J=2 Hz, 1H), 6.2 (d, J=2 Hz, 1H), 6.72-6.98 (m, 2H), 7.16 (s, 2H), 7.2-7.6 (m, 20H), 9.3 (s, 1H).

¹³C NMR (75 MHz, CDCl₃) δ=26.92, 66.94, 69.06, 69.23, 70.17, 70.26, 74.91, 75.63, 92.3, 95.27, 107.05, 113.6, 113.9, 119.7, 126.79, 127.32, 127.45, 127.51127.58, 127.59, 127.63, 128.2, 128.24, 128.29, 137.18, 137.29, 137.38, 137.4, 147.17, 147.76, 157.06, 157.71, 157.75.

MS=651.5 [M⁺+H]

[α]²⁰_{D}=−1.437° (c=1, CH₂Cl₂/MeOH, 3/1, v/v)

### Example 15

### Preparation of 5, 7, 3′, 4′-Tetra-O-benzyl-(−)-catechin

Part A—Preparation of 5,7,3′,4′-Tetra-O-(−)-catechin-3-O-propyl ester.

To a suspension of the (1R,2R)-3-(2,4-bis(benzyloxy)-6-hydroxyphenyl)-1-(3′,4′-bis(benzyloxy)phenyl)propane-1,2-diol from Example 12 (40.4 g, 60.47 mmol, 1 eq) in 1,2-dichloroethane (750 mL) was added triethylorthopropionate (12.76 g, 108.8 mmol, 1.8 eq) followed by pyridinium para-toluenesulfonate (8.2 g, 32.65 mmol, 0.54 eq) with stirring. The mixture was then heated at 60°-62° C. (internal temperature) and maintained at this temperature for 3 to 4 h until TLC indicated consumption of the starting material. The reaction mixture was then cooled to RT and passed through a plug of silica gel (300 g). The silica gel plug was further washed with dichloromethane (1.5 L). The filtrates were combined and the solvent was removed in vacuo to give compound 5,7,3′,4′-tetra-O-(−)-catechin-3-O-propyl ester. The yield was 41.9 g (91%). HPLC purity=98.1% (AUC). Chiral HPLC=86% ee.

¹H NMR (300 MHz, CDCl₃) δ=0.98 (t, J=7.6 Hz, 3H), 2.0-2.28 (m, 2H), 2.69 (dd, J=6.8, 16.8 Hz, 1H), 2.82 (dd, J=5.4, 16.8 Hz, 1H), 4.98 (s, 4H), 5.08 (s, 2H), 5.1 (s, 2H), 5.24-5.3 (m, 1H), 6.25 (d, J=1.9 Hz, 2H), 6.88 (s, 2H), 6.94 (s, 1H), 7.17-7.46 (m, 20H).

¹³C NMR (75 MHz, CDCl₃) δ=8.97, 24.15, 30.34, 43.44, 68.86, 69.98, 70.15, 71.29, 71.33, 78.36, 93.79, 94.48, 101.47, 113.59, 114.98, 120.0, 127.14, 127.25, 127.45, 127.54, 127.76, 127.91, 127.99, 128.16, 128.44, 128.45, 128.53, 128.59, 129.79, 131.17, 136.88, 136.9, 137.12, 148.92, 148.95, 154.93, 157.68, 158.63, 173.49.

MS=707.3 [M⁺+H]

[α]²⁰_{D}=−0.881° (c=1, CH₂Cl₂)

Part B:—Conversion of 5,7,3′,4′-Tetra-O-(−)-catechin-3-O-propyl ester to 5,7,3′,4′-Tetra-O-benzyl-(−)-Catechin.

The crude 5,7,3′,4′-Tetra-O-(−)-catechin-3-O-propyl ester was dissolved in a mixture of dichloromethane (500 mL) and methanol (250 mL) followed by the addition of potassium carbonate (12.5 g, 90.7 mol, 1.5 eq). The reaction mixture was stirred at RT for 3 to 4 h until TLC indicated that the reaction was complete. The reaction mixture was filtered. The solvent was removed. The crude product was dissolved in methanol (500 mL) and stirred at RT for 0.5 h. The solids were suction filtered and washed with methanol (1×200 mL) and dried in vacuo at RT to give crude 5,7,3′,4′-tetra-O-benzyl-(−)-catechin. The yield was 38.4 g (97.5%).

Crude 5,7,3′,4′-tetra-O-benzyl-(−)-catechin (37.5 g) was dissolved in toluene (2.7 L) at ˜40° C. (bath temperature). The solution obtained was allowed to stand at RT for ˜40 h. The solids were suction filtered. The filtrate was concentrated in vacuo to give the desired compound (31.4 g, 84%) with 91% ee as judged by chiral HPLC. The solid was again dissolved in toluene (1.4 L), warmed to 40-45° C. (bath temperature), and then allowed to stand at RT for 15 h. The solids were suction filtered. The filtrate was concentrated in vacuo to give the desired compound as an off-white solid. The yield was 21.6 g (61%). HPLC purity was 100% (AUC). Chiral HPLC was 96% ee.

¹H NMR (300 MHz, CDCl₃) δ=1.55 (d, J=3.7 Hz, 1H), 2.62 (dd, J=8.9, 16.9 Hz, 1H), 3.08 (dd, J=5.7, 16.5 Hz, 1H), 3.8-4.02 (m, 1H), 4.6 (d, J=8.2 Hz, 1H), 4.9 (s, 2H), 4.97 (s, 2H), 5.04 (s, 4H), 6.15 (d, J=2.3 Hz, 1H), 6.22 (d, J=2.3 Hz, 1H), 6.85 (s, 2H), 7.0 (s, 1H), 7.15-7.4 (m, 20H). ¹³C NMR (75 MHz, CDCl₃) δ=31.88, 68.19, 69.95, 70.15, 71.32, 71.37, 81.59, 93.89, 94.48, 102.34, 114.03, 115.12, 120.61, 127.13, 127.23, 127.6, 127.8, 127.91, 127.97, 128.48, 128.128.62, 128.78, 130.01, 136.93, 136.96, 137.04, 137.28, 149.15, 149.41, 155.32, 159.81.

MS=651.5 [M⁺+H]

[α]²⁰_{D}=−0.508° (c=1, CH₂Cl₂)

### Example 16

### Preparation of 5,7,3′,4′-Tetra-O-benzyl-(+)-epicatechin

Part A—Preparation of (2S)-5,7-Bis(benzyloxy)-2-(3′,4′-bis(benzyloxy)-chroman-3-one

To a solution of the 5,7,3′,4′-tetra-O-benzyl-(−)-catechin from Example 13 (9.36 g, 14.4 mmol, 1 eq) in dichloromethane (200 mL) was added Dess-Martin periodinane reagent (7.15 g, 16.87 mol, 1.17 eq). A clear solution was obtained after stirring for 5 min. To this wet dichloromethane (10 mL) was added dropwise. The resulting reaction mixture was stirred at RT for ˜2.5 h, until TLC indicated that the reaction was complete. The reaction mixture was quenched with 10% aqueous sodium bicarbonate solution (100 mL). The organic layer was separated. The aqueous layer was extracted with dichloromethane (1×500 mL, 1×200 mL). The organic layers were combined, washed with water (1×300 mL), dried over sodium sulfate, and filtered. The solvent was removed in vacuo. The crude product was dissolved in dichloromethane (25 mL) and passed through a silica gel plug (75 g). The silica gel plug was eluted with dichloromethane (300 mL). The combined filtrate was concentrated in vacuo to give the desired compound as an off-white solid. The yield was 7.85 g (85%). HPLC purity was 88% (AUC).

¹H NMR (300 MHz, CDCl₃) δ=3.31-3.64 (m, 2H), 4.96 (s, 4H), 5.07 (s, 2H), 5.1 (s, 2H), 5.2 (s, 1H), 6.3 (d, J=2 Hz, 2H), 6.5 (s, 2H), 6.94 (s, 1H), 7.1-7.52 (m, 20H).

¹³C NMR (75 MHz, CDCl₃) δ=33.66, 70.14, 70.26, 71.21, 71.31, 83.04, 95.13, 95.91, 102.01, 113.54, 114.86, 120.01, 126.7, 127.19, 127.41, 127.61, 127.8, 128.05, 128.08, 128.24, 128.44, 128.47, 128.58, 128.62, 136.49, 136.64, 137.02, 137.15, 148.48, 149.25, 154.56, 157.07, 159.49, 207.22.

MS=649.5 [M⁺+H]

Part B—Conversion of (2S)-5,7-Bis(benzyloxy)chroman-3-one to 5,7,3′,4′-Tetra-O-benzyl-(+)-epicatechin.

A suspension of the compound from Part A (6 g, 9.26 mmol, 1 eq) in toluene (90 mL) and 2-propanol (33 mL) was heated at reflux with stirring while connected to a distillation setup to collect the acetone formed during the reaction. The reaction was continued until TLC indicated the reaction was complete. The mixture was cooled to RT and quenched with 5% aqueous sulfuric acid (125 mL) with stirring. The reaction mixture was extracted with ethyl acetate (2×150 mL). The organic layers were combined and washed with water (3×100 mL), dried over sodium sulfate, and filtered. The solvent was removed in vacuo. The crude product was recrystallized with benzene/heptane (4/1, v/v, 250 mL) to give the desired product as an off-white solid. The yield was 5.38 g (89%). HPLC purity was 100% (AUC). Chiral HPLC was 96% ee.

¹H NMR (300 MHz, CDCl₃) δ=1.65 (br s, 1H), 2.8-3.04 (m, 2H), 4.18 (br s, 1H), 4.88 (s, 1H), 5.0 (s, 4H), 5.2 (s, 4H), 6.2 (s, 2H), 6.92 (s, 2H), 7.13 (s, 1H), 77.2-7.6 (m, 20H).

¹³C NMR (75 MHz, CDCl₃) δ=28.26, 66.74, 70.01, 70.2, 71.43, 71.5, 78.42, 94.14, 94.82, 101.06, 113.72, 116.24, 119.57, 127.22, 127.3, 127.53, 127.82, 127.86, 127.88, 127.97, 128.48, 128.65, 128.88, 128.91, 131.56, 136.88, 137.85, 149.03, 158.87, 158.83

MS=651.5 [M⁺+H]

[α]²⁰_{D}=+2.4° (c=1, Acetone)

### Example 17

### Debenzylation of (−)-Catechin

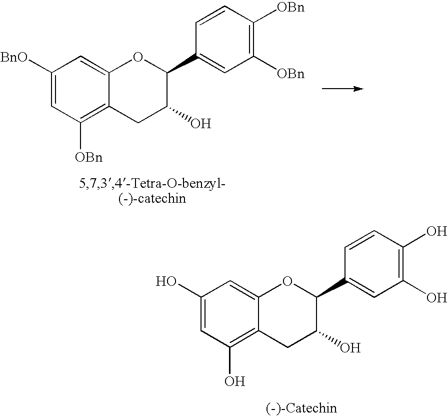

A suspension of 5,7,3′,4′-tetra-O-benzyl-(−)-catechin (2.13 g, 3.27 mmol, 1 eq) and 20% palladium hydroxide on carbon (50% wet, 0.53 g, 25 wt. %) in ethyl acetate (125 mL) was hydrogenated at RT at ˜15 psi for 3 h.

HPLC indicated consumption of the starting material. The catalyst was removed by filtration through a 0.45-micron cartridge. The cartridge was washed with ethyl acetate (20 mL). The combined filtrate was concentrated in vacuo. The residue was dissolved in water (100 mL), frozen and lyophilized to give the desired compound as a white solid. The yield was 0.8 g (84%). HPLC purity was 99% (AUC).

¹H NMR (300 MHz, Acetone-d₆) δ=2.51 (dd, 1H, J=8.3, 16 Hz), 2.9 (dd, 1H, J=5.4, 16 Hz), 3.78-4.05 (m, 2H), 4.58 (d, 1H, J=7.6 Hz), 5.88 (d, 1H, J=2.3 Hz), 6.02 (d, 1H, J=2.3 Hz), 6.6-6.8 (m, 2H), 6.86 (d, 1H, J=1.7 Hz), 7.8 (d, 2H, J=16.6 Hz), 7.91 (s, 1H), 8.1 (s, 1H).

¹³C NMR (75 MHz, Acetone-d₆) δ=28.76, 68.37, 82.68, 95.5, 96.18, 100.67, 115.25, 115.75, 120.08, 132.22, 145.69, 156.91, 157.19, 157.71.

MS=291.1 [M⁺+H]

### Example 18

### Debenzylation of (+)-Epicatechin

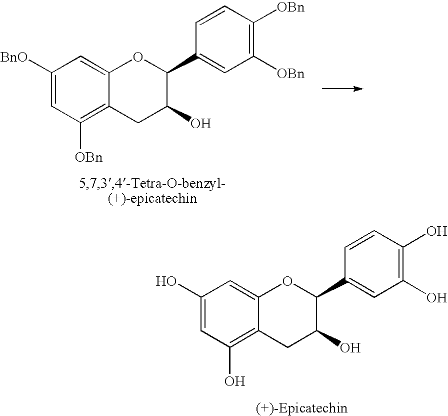

A suspension of 5,7,3′,4′-tetra-O-benzyl-(+)-epicatechin (0.4 g, 0.615 mmol. 1 eq.) and 20% palladium hydroxide on carbon (50% wet, 0.0.08 g, 25 wt. %) in ethyl acetate (20 mL) was hydrogenated at RT at ˜15 psi for 3 h. HPLC indicated the consumption of the starting material. The catalyst was removed by filtration through a 0.45-micron cartridge. The cartridge was washed with ethyl acetate (10 mL). The combined filtrate was concentrated in vacuo. The residue was dissolved in water (100 mL), frozen and lyophilized to give the desired compound as a white solid. The yield was 0.18 g, 84%. HPLC purity was 98.4% (AUC).

¹H NMR (300 MHz, Acetone-d₆) δ=2.44 (dd, 1H, J=3.3, 16.5 Hz), 2.68 (dd, 1H, J=4.5, 16.5 Hz), 3.3 (s, 1H), 3.0-4.02 (m, 1H), 4.6 (d, 1H, J=4.6 Hz), 4.7 (s, 1H), 5.68 (d, 1H, J=2.2 Hz), 5.85 (d, 1H, J=2.2 Hz), 8.67 (s, 1H), 8.75 (s, 1H), 8.88 (s, 1H), 9.1 (s, 1H).

¹³C NMR (75 MHz, Acetone-d₆) δ=28.07, 64.82, 77.96, 94.0, 95.0, 98.4, 114.66, 114.8, 117.85, 130.51, 144.34, 144.4, 155.66, 156.13, 156.41.

MS=291.1 [M⁺+H]

While the invention has been described with respect to certain specific embodiments, it will be appreciated that many modifications and changes may be made by those skilled in the art without departing from the invention. It is intended, therefore, for the appended claims to cover all such modifications and changes as may fall within the true spirit and scope of the invention.

What is claimed is:

1. A process for preparing a racemic mixture consisting essentially of 5,7,3′,4′-tetra-O-benzyl-(±)-catechin as the major diastereomer and 5,7,3′,4′-tetra-O-benzyl-(±)-epicatechin as the minor diastereomer comprises the steps of:
(a) condensing 2-hydroxy-4,6-bis(benzyloxy)-acetophenone with 3,4-bis(benzyloxy)benzaldehyde in the presence of a base to form (E)-1-(2′,4′-bis(benzyloxy)-6-hydroxyphenyl-3-(3′,4′-bis(benzyloxy)phenyl)prop-2-en-1-one;
(b) cyclizing the compound formed in step (a) under reductive conditions to form 5,7-bis(benzyloxy)-2-(3′,4′-bis(benzyloxy)phenyl-2H-chromene; and
(c) oxidizing the compound from step (b) to form the racemic mixture.

2. The process of claim 1, further comprising the step of preparing the 2-hydroxy-4,6-bis(benzyloxy)-acetophenone by benzylating 2,4,6-trihydroxy-acetophenone with benzyl bromide or benzyl chloride in N,N-dimethylformamide in the presence of potassium carbonate at from room temperature to about 80° C.; further comprising the step of preparing the 3,4-bis(benzyloxy)benzaldehyde by benzylating 3,4-benzylaldehyde with benzyl bromide or benzyl chloride in N,N-dimethylformamide in the presence of potassium carbonate; and further comprising the step of separating the epimers in the racemic mixture by chemical resolution or by preparative high pressure liquid chromatography.

3. The process of claim 2, further comprising the step of debenzylating the epimers with excess palladium hydroxide in ethyl acetate under hydrogen atmosphere at room temperature for about 2 to about 3 hours.

4. The process of claim 2, further comprising the steps of (a) oxidizing the 5,7,3′,4′-tetra-O-benzyl-(+)-catechin or the 5,7,3′,4′-tetra-O-benzyl-(−)-catechin with Dess Martin Periodinane to form (2S)- or (2R)-5,7-bis(benzyloxy)-2-3′,4′-bis(benzyloxy)-chroman-3-one and (b) stereoselectively reducing the (2S)- or (2R)-5,7-bis(benzyloxy)-2-3′,4′-bis(benzyloxy))-chroman-3-one from step (a) with aluminum isopropoxide and 2-propanol in toluene at reflux to form 5,7,3′,4′-tetra-O-benzyl-(+)-epicatechin or 5,7,3′,4′-tetra-O-benzyl-(−)-epicatechin.

5. The process of claim 4, further comprising the step of debenzylating the 5,7,3′,4′-tetra-O-benzyl-(+)-catechin, 5,7,3′,4′-tetra-O-benzyl-(−)-catechin, the 5,7,3′,4′-tetra-O-benzyl-(−)-epicatechin, 5,7,3′,4′-tetra-O-benzyl-(+)-epicatechin with palladium hydroxide under a hydrogen atmosphere in ethyl acetate at room temperature.

6. An improved process for preparing (E)-1-(2′,4′-bis(benzyloxy)-6-hydroxyphenyl-3-(3′,4′-bis(benzyloxy)phenyl)prop-2-en-1-one comprises condensing 2-hydroxy-4,6-bis(benzyloxy)-acetophenone with 3,4-bis(benzyloxy)-benzaldehyde in the presence of a base followed by reaction with sodium borohydride and cerium heptahydrate at a low temperature in a solution of ethanol and tetrahydrofuran.

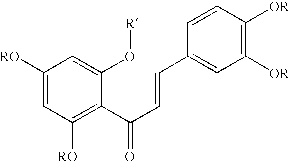

7. 5,7-Bis(benzyloxy)-2-(3′,4′-bis(benzyloxy)phenyl-2H-chromene.

8. A process for preparing the 5,7-bis(benzyloxy)-2-(3′,4′-bis-(benzyloxy)phenyl-2H-chromene of claim 7 comprises the step of cyclizing (E)-1-(2′,4′-bis(benzyloxy)-6-hydroxyphenyl-3-(3′,4′-bis(benzyloxy)-phenyl)prop-2-en-1-one under reductive conditions using sodium borohydride in refluxing ethanol.

9. A process for preparing a racemic mixture consisting essentially of 5,7,3′,4′-tetra-O-benzyl-(±)-catechin as the major diastereomers and 5,7,3′,4′-tetra-O-benzyl-(±)-epicatechin as the minor diastereomers comprises the steps of:
(a) dihydroxylating 5,7-bis(benzyloxy)-2-(3′,4′-bis(benzyloxy)phenyl-2H-chromene to form racemic (3S,4S)-5,7-bis(benzyloxy)-2-(3′,4′-bis(benzyloxy)-phenyl)chroman-3,4-diol; and
(b) reducing the 3,4-diol from step (a) to form the racemic mixture.

10. The process of claim 9, wherein dihydroxylating step (a) is carried out with osmium tetraoxide and N-methyl-morpholine N-oxide in a mixture of tert-butanol, water, and tetrahydrofuran at room temperature; and wherein reducing step (b) is carried out with sodium cyanoborohydride in acetic acid.

11. The process of claim 10, further comprising the step of separating the diastereomers and debenzylating the separated epimers by reaction with excess palladium hydroxide in ethyl acetate under hydrogen atmosphere at room temperature for about 1 to about 3 hours.

12. A process for preparing (±) 5,7,3′,4′-tetra-O-benzyl-(+)-catechin and 5,7,3′,4′-tetra-O-(±)-epicatechin comprises the steps of:
(a) coupling 3,5-bis(benzyloxy)phenol with (E)-3-(3′,4′-bis(benzyloxy)-phenyl)prop-2-ene-1-ol under acidic conditions to form (E)-3,5-bis(benzyloxy)-2-(3′,4′-bis(benzyloxy)phenyl)allyl)phenol;
(b) reacting the compound formed in step (a) with tert-butyldimethylsilane chloride to form (E)-(3,5-bis(benzyloxy)-2-(3-(3′,4′-bis(benzyloxy)phenyl)allyl)-phenoxy)(tert-butyl)dimethysilane;
(c) dihydroxylating the compound formed in step (b) using osmium tetraoxide and N-methyl morphiline N-oxide in a mixture of tert-butanol, water, and tetrahydrofuran at room temperature to form 3,5-bis(benzyloxy)-2-(5-(3′,4′-bis-(benzyloxy)phenyl)-2-ethoxy-1,3-dioxolane-4-yl)phenol;
(d) removing the (tert-butyl)dimethylsilane protecting group from the compound of step (d) to form 3-(2,4-bis(benzyloxy)-6-(hydroxyphenyl)-1-(3′,4′-bis(benzyloxy)phenyl)propane-1,2-diol;
(e) activating the compound from step (d) by reaction with triethylorthoformate or triethylorthopropionate to form 3,5-bis(benzyloxy)-2-(5-(3′,4′-bis(benzyloxy)phenyl)-2-ethoxy-1,3-dioxolan-4-yl)phenol from the orthoformate or 3,5-bis(benzyloxy)-2-(5-(3′,4′-bis(benzyloxy)phenyl-2-ethoxy-2-ethyl-1,3-dioxolan-4-yl)propyl)phenol from the orthopropionate; and
(f) reacting the diol from step (e) with potassium carbonate in a mixture of methanol and dichloromethane or dichloroethane at room temperature or at 60° C. to form 5,7,3′,4′-tetra-O-benzyl-(±)-catechin.

13. The process of claim 12, further comprising the steps of removing the solvent from the mixture from step (g) under vacuum; extracting the residue with ethyl acetate and water; removing the water from the extract; drying the ethyl acetate over sodium sulfate; and evaporating the ethyl acetate to recover the crude 5,7,3′4′-tetra-O-benzyl-(±)-catechin.

14. The process of claim 13, wherein the debenzylating step is carried out using palladium hydroxide in ethyl acetate at room temperature under hydrogen atmosphere using a balloon.

15. The process of claim 12, further comprising the step of separating the diastereomers and debenzylating the separated epimers by reaction with palladium hydroxide in ethyl acetate at room temperature under hydrogen atmosphere.

16. A process for preparing 5,7,3′,4′-tetra-O-benzyl-(−)-catechin or 5,7,3′,4′-tetra-O-benzyl-(+)-epicatechin comprises the steps of:
(a) condensing 2-hydroxy-4,6-bis(benzyloxy)acetophenone with 3,4-bis(benzyloxy)benzaldehyde in the presence of a base in N,N-dimethylformamide to form (E)-1-(2,4-bis(benzyloxy)-6-hydroxyphenyl-3-(3′,4′-bis(benzyloxy)phenyl)prop-2-en-1-one;
(b) selectively reducing the compound formed in step (a) with sodium borohydride and cerium chloride heptahydrate in a mixture of tetrahydrofuran and ethanol to form (E)-3,5-bis(benzyloxy)-2-(3′,4′-bis(benzyloxy)phenyl)allyl)phenol;
(c) reacting the compound formed in step (b) with tert-butyldimethylsilane chloride in imidazole and dimethylformamide or in the presence of triethylamine and N,N-dimethylaminopyridine in dichloromethane at room temperature to form (E)-(3,5-bis(benzyloxy)-(3-3′,4′-bis(benzyloxy)phenyl)-allyl)-phenoxy)(tert-butyl)dimethylsilane;
(d) reacting the compound formed in step (c), in the presence of methanesulfonamide in a mixture of tert-butanol, water, and tetrahydrofuran or dichloromethane with AD-mix-β to form (1R,2R)-3-(2,4-bis(benzyloxy)-6-tert-butyldimethylsiloxy)phenyl-1-(3′,4′-bis(benzyloxy)phenyl)propene-1,2-diol or with AD-mix-α to form (1S,2S)-3-(2,4-bis(benzyloxy)-6-tert-butyldimethylsiloxy)phenyl-1-(3′,4′-bis(benzyloxy)phenyl)propene-(1,2-diol);
(e) reacting the (1R,2R)- or (1S,2S)-1,2-diol formed in step (d) with n-tetrabutylammonium fluoride in acetic acid and tetrahydrofuran or dichloromethane to form (1R,2R)- or (1S,2S)-3-(2,4-bis(benzyloxy)-6-hydroxyphenyl)-1-(3′,4′-bis(benzyloxy)phenyl-propane-1,2-diol;
(f) reacting the (1S,2S)- or (1R,2R)-1,2-diol formed in step (e) with triethylorthopropionate and pyridinium p-toluenesulfonate to form triethylorthopropionate or triethylorthoformate, to form 5,7,3′,4′-tetra-O-benzyl-(−)-catechin-3-O-propyl ester; and
(g) reacting the compound formed in step (f) 5,7,3′,4′-tetra-O-benzyl-(−)-catechin-3-O-propyl ester with potassium carbonate in a mixture of methanol and dichloromethane or dichloroethane to form the 5,7,3′,4′-tetra-O-benzyl-(−)-catechin or the 5,7,3′,4′-tetra-O-benzyl-(+)-catechin; and
(h) optionally separating the 5,7,3′,4′-tetra-O-benzyl-(−)-catechin or 5,7,3′,4′-tetra-O-benzyl-(+)-catechin and debenzylating the separated compounds by reaction with palladium hydroxide in ethyl acetate at room temperature.

17. The process of claim 16, wherein the debenzylation is carried out using palladium hydroxide in ethyl acetate at room temperature under hydrogen atmosphere using a balloon to form (−)-catechin or (+)-catechin.

18. A process for chemically resolving a racemic mixture of 5,7,3′,4′-tetra-O-benzyl-(±)-catechin and 5,7,3′,4′-(±)-epicatechin comprises the steps of:
(a) esterifying the 3-position of the compounds in the racemic mixture with dibenzoyl-L-tartaric acid monomethyl ester to form racemic (±)-(2R,3R)-1-((2R,3S)-5,7-bis(benzoyloxy)-2-(3′,4′-bis(benzyloxy)phenyl)chroman-3-yl)-4-methyl-2,3-bis(benzyloxy)succinate;
(b) fractionally crystallizing the compounds from step (a) to recover enantiomerically pure (+)-2R,3R)-1-((2R,3S)-5,7-bis(benzolyloxy)-2-3′,4′-bis(benzyl)oxy)phenyl)chroman-3-yl)-4-methyl-2,3-bis(benzyloxy)succinate; and
(c) hydrolyzing the compound from step (b) in a solution of about 80% dichloromethane and about 20% heptane (v/v) with 0.05 M of potassium hydroxide in methanol and dichloromethane at about 40° to about 45° C. to form the enantiomerically pure 5,7,3′,4′-tetra-O-benzyl-(+)-catechin.

19. The process of claim 19, further comprising the steps of preparing 2-hydroxy-4,6-bis(benzyloxy)-acetophenone by benzylating 2,4,6-trihydroxy-acetophenone with benzyl bromide or benzyl chloride in N,N-dimethylformamide in the presence of potassium carbonate at from room temperature to about 80° C. and preparing the 3,4-bis(benzyloxy)benzaldehyde by benzylating 3,4-benzylaldehyde with benzyl bromide or benzyl chloride in N,N-dimethylformamide in the presence of potassium carbonate.

20. A process for the selective reduction of (E)-1-(2,4-bis(benzyloxy)-6-hydroxyphenyl-3-(3′,4′-bis(benzyloxy)phenyl)prop-2-en-1-one comprises the step of carrying out the reduction with sodium borohydride and cerium chloride at about 0° C. to about 5° C. in a mixture of tetrahydrofuran and ethanol.
